(19) United States
(12) Patent Application Publication
(10) Pub. No.: US 2024/0309442 A1
(43) Pub. Date: Sep. 19, 2024

(54) KIT AND METHOD FOR EMBRYO IMPLANTATION

(71) Applicant: Inti Taiwan, Inc., Hsinchu County (TW)

(72) Inventors: Shih-Ting Kang, Hsinchu County (TW); Wei-Ming Chen, Hsinchu County (TW)

(73) Assignee: Inti Taiwan, Inc., Hsinchu County (TW)

(21) Appl. No.: 18/733,895

(22) Filed: Jun. 5, 2024

Related U.S. Application Data
(62) Division of application No. 16/914,040, filed on Jun. 26, 2020.
(60) Provisional application No. 62/869,574, filed on Jul. 2, 2019.

(51) Int. Cl. C12Q 1/686; C12Q 1/6883; G06F 17/18

(52) U.S. Cl. CPC C12Q 1/686; C12Q 1/6883; C12Q 2600/112; C12Q 2600/178; G06F 17/18

(57) ABSTRACT

The disclosure relates to methods for determining an endometrial status using a sample, for example, an endometrial biopsy, from a woman, comprising: (a) performing an assay on the endometrial sample from the woman to determine a microRNA (miRNA) expression profile of the endometrial sample, wherein the miRNA expression profile comprises expression levels of a plurality of miRNAs, for example, 167 miRNAs having the sequences of SEQ ID NOs:1-167, respectively; and (b) analyzing the miRNA expression profile to obtain a receptivity predictive score using, for example, a computer-based algorithm. Aspects of the disclosure further relate to kits suitable for performing the methods, as well as uses of the kits for diagnostic and therapeutic purposes.

### CROSS-REFERENCE TO RELATED APPLICATION

This application is a divisional application of and claims the priority benefit of a prior application Ser. No. 16/914,040, filed on Jun. 26, 2020, now pending. The prior application Ser. No. 16/914,040 claims the priority benefit of U.S. Provisional Application No. 62/869,574, filed on Jul. 2, 2019. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on May 20, 2024, is named 087210-1-US-sequence listing and is 212,245 bytes in size.

### BACKGROUND

### Technical Field

The disclosure relates to methods for determining the endometrial receptivity of a woman using (a) a microRNA (miRNA) expression profile comprising expression levels of a plurality of miRNAs, for example, 167 miRNAs, and (b) a computer-based algorithm that classifies the endometrial status of the woman based on the miRNA expression profile. Aspects of the disclosure further relate to kits suitable for performing the methods, as well as uses of the kits for diagnostic and therapeutic purposes. In some embodiments, the methods and/or kits are used to classify a woman's responsiveness to an in vitro fertilization (IVF) treatment.

### Description of Related Art

Assisted reproductive technologies, including IVF, emerged as potential approaches to address a lack of reproductive success. A major factor in the success rates of IVF is the receptive state of the endometrium. An endometrium is receptive only for a relatively short period referred to as window of implantation (WOI). This usually occurs around days 19-21 of the menstrual cycle. There is a longstanding need for monitoring the status of the endometrium not only based on the calendar approach, which tends to be unreliable, but also directly by examining the endometrium itself, which would indicate the opportunity for embryo implantation in a more reliable way.

Human endometrium is a tissue cyclically regulated by both proteins and miRNAs. The human genome comprises more than 2500 miRNAs, some of which have been shown to play roles in reproductive cycles. For example, recent literature demonstrated that certain miRNAs regulate the expression of genes involved in the establishment and progression of WOI.

Traditionally, histological and imaging methods were used to assess the status of the endometrium. However, it was long recognized that they are time consuming and often cannot clearly distinguish between the receptive and non-receptive states of the endometrium. Methods based on the examination of gene expression levels have also been developed. Early studies focused on a few marker genes. Igenomix developed an “Endometrial Receptivity Analysis” (ERA) test, relying on a microarray of specific 238 genes involved in endometrial receptivity. However, microarray-based ERA test has certain drawbacks. For example, it is known that microarray-based gene expression measurements require significant amounts of tissue samples. In addition, microarray technology generally has lower specificity as compared to quantitative polymerase chain reaction (qPCR) technology. Next-generation sequencing (NGS)-based ERA test is only emerging.

Thus, there remains a need for improved methods of determining endometrial receptivity that require less tissue input and/or provide more reliable determination of the receptive or non-receptive status of the endometrium in a woman.

### SUMMARY

The disclosure relates to methods for determining endometrial receptivity using a sample, for example, an endometrial biopsy, from a woman, comprising: (a) performing an assay on the endometrial sample from the woman to determine a miRNA expression profile of the endometrial sample, wherein the miRNA expression profile comprises expression levels of a plurality of miRNAs, for example, 167 miRNAs having the sequences of SEQ ID NOs:1-167, respectively; and (b) analyzing the miRNA expression profile to obtain a receptivity predictive score, wherein the receptivity predictive score determines the woman's endometrial receptivity status. Aspects of the disclosure further relate to kits suitable for performing the methods, as well as uses of the kits for determining an endometrial status of a woman.

Certain embodiments of the present disclosure are summarized in the following paragraphs. This list is only exemplary and not exhaustive of all of the embodiments provided by this disclosure.

### EMBODIMENT 1

A method of determining an endometrial status, comprising: (a) performing an assay on an endometrial sample from a woman to determine a miRNA expression profile of the endometrial sample, wherein the miRNA expression profile comprises expression levels of a plurality of miRNAs; and (b) analyzing the miRNA expression profile to obtain a receptivity predictive score, wherein the receptivity predictive score classifies the endometrial status of the woman, and wherein the endometrial status comprises a pre-receptive state, a receptive state, or a post-receptive state, and wherein the plurality of miRNAs comprise at least 50, 75, 100, 125, 150, or 200 miRNAs, and preferably at least 167 miRNAs having the sequences of SEQ ID NOs: 1-167, respectively.

### EMBODIMENT 2

The method of embodiment 1, wherein the endometrial sample is obtained from the uterine cavity of the woman.

### EMBODIMENT 3

The method of embodiment 1 or embodiment 2, wherein the endometrial sample comprises an endometrial biopsy, an endometrial lavage, or combination thereof.

### EMBODIMENT 4

The method of any one of embodiments 1-3, wherein the endometrial sample is obtained (i) seven days after an endogenous luteinizing hormone (LH) surge in the woman or (ii) five days after a progesterone administration in the woman.

### EMBODIMENT 5

The method of any one of embodiments 1-4, wherein the miRNA expression profile is determined by qPCR, sequencing, microarray, or RNA-DNA hybrid capture technology.

### EMBODIMENT 6

The method of embodiment 5, wherein the miRNA expression profile is determined by qPCR performed on a cDNA preparation synthesized from the miRNAs in the endometrial sample.

### EMBODIMENT 7

The method of embodiment 6, wherein the cDNA synthesis is performed using a universal reverse transcription primer having a nucleotide sequence represented by the following general formula: 5′-R-(dT)nVN-3′, wherein R comprises SEQ ID NO:168, (dT)n is an n number of continuous thymine residues, wherein n is 19, V is an adenine residue, a guanine residue, or a cytosine residue, and N is an adenine residue, a guanine residue, a cytosine residue, or a thymine residue.

### EMBODIMENT 8

The method of any one of embodiments 1-7, wherein the receptivity predictive score is a value produced by a computer-based algorithm and calculated using the equation of MIRA score=f(X ∈eq(C))=Xβ+ϵ, β being a vector of coefficients, and ϵ being an error.

### EMBODIMENT 9

The method of embodiment 8, where the computer-based algorithm is established by performing one or more of the following steps: data normalization, data scaling, data transformation, prediction modeling, and cross-validation.

### EMBODIMENT 10

The method of embodiment 8 or embodiment 9, wherein a receptivity predictive score greater than 1 indicates the pre-receptive state, a receptivity predictive score less than −1 indicates the post-receptive state, and a receptivity predictive score from −1 to 1 indicates the receptive state.

### EMBODIMENT 11

The method of any one of embodiments 1-10, wherein if the endometrial status is determined to be at the pre-receptive state or the post-receptive state, further comprising: repeating steps (a) and (b) at least once or until the endometrial status is determined to be at the receptive state.

### EMBODIMENT 12

The method of any one of embodiments 1-11, wherein the woman suffers or suffered from an implantation failure.

### EMBODIMENT 13

The method of any one of embodiments 1-12, wherein the woman is subject to an IVF treatment.

### EMBODIMENT 14

The method of embodiment 13, wherein the receptivity predictive score further classifies the woman's responsiveness to the IVF treatment.

### EMBODIMENT 15

A method of detecting endometrial receptivity for embryo implantation in a woman, comprising: (a) performing an assay on an endometrial sample from the woman to determine a miRNA expression profile of the endometrial sample, wherein the miRNA expression profile comprises expression levels of a plurality of miRNAs; and (b) analyzing the miRNA expression profile to obtain a receptivity predictive score, wherein the receptivity predictive score determines whether the woman has endometrial receptivity for embryo implantation, and wherein the plurality of miRNAs comprise at least 50, 75, 100, 125, 150, or 200 miRNAs, and preferably at least 167 miRNAs having the sequences of SEQ ID NOs:1-167, respectively.

### EMBODIMENT 16

The method of embodiment 15, wherein the endometrial sample is obtained from the uterine cavity of the woman.

### EMBODIMENT 17

The method of embodiment 15 or embodiment 16, wherein the endometrial sample comprises an endometrial biopsy, an endometrial lavage, or combination thereof.

### EMBODIMENT 18

The method of any one of embodiments 15-17, wherein the endometrial sample is obtained (i) seven days after an endogenous luteinizing hormone (LH) surge in the woman or (ii) five days after a progesterone administration in the woman.

### EMBODIMENT 19

The method of any one of embodiments 15-18, wherein the miRNA expression profile is determined by qPCR, sequencing, microarray, or RNA-DNA hybrid capture technology.

### EMBODIMENT 20

The method of embodiment 19, wherein the miRNA expression profile is determined by qPCR performed on a cDNA preparation synthesized from the miRNAs in the endometrial sample.

### EMBODIMENT 21

The method of embodiment 20, wherein the cDNA synthesis is performed using a universal reverse transcription primer having a nucleotide sequence represented by the following general formula: 5′-R-(dT)nVN-3′, wherein R comprises SEQ ID NO:168, (dT)n is an n number of continuous thymine residues, n is 19, V is an adenine residue, a guanine residue, or a cytosine residue, and N is an adenine residue, a guanine residue, a cytosine residue, or a thymine residue.

### EMBODIMENT 22

The method of any one of embodiments 15-21, wherein the receptivity predictive score is a value produced by a computer-based algorithm and calculated using the equation of MIRA score=f(X∈eq(C))=Xβ+ϵ, β being a vector of coefficients, and & being an error.

### EMBODIMENT 23

The method of embodiment 22, where the computer-based algorithm is established by performing one or more of the following steps: data normalization, data scaling, data transformation, prediction modeling, and cross-validation.

### EMBODIMENT 24

The method of embodiment 22 or embodiment 23, wherein a receptivity predictive score from −1 to 1 indicates that the woman has endometrial receptivity for embryo implantation.

### EMBODIMENT 25

The method of any one of embodiments 15-24, wherein the woman suffers or suffered from an implantation failure.

### EMBODIMENT 26

A kit comprising: (a) one or more miRNA profiling chips targeting a plurality of miRNAs, and (b) instructions on (i) determining a miRNA expression profile of an endometrial sample from a woman, optionally using the one or more miRNA profiling chips, and (ii) obtaining a receptivity predictive score based on the miRNA expression profile, using a computer-based algorithm, wherein the plurality of miRNAs comprise at least 50, 75, 100, 125, 150, or 200 miRNAs, and preferably at least 167 miRNAs having the sequences of SEQ ID NOs:1-167, respectively.

### EMBODIMENT 27

The kit of embodiment 26, wherein the one or more miRNA profiling chips comprise primers for detection of expression levels of the plurality of miRNAs.

### EMBODIMENT 28

The kit of embodiment 27, wherein the miRNA profiling chips are suitable for performing a qPCR, sequencing, microarray, or RNA-DNA hybrid capture assay, preferably qPCR, to detect the expression levels of the plurality of miRNAs.

### EMBODIMENT 29

Use of the kit of embodiment 27 or embodiment 28 for determining an endometrial status of a woman.

### EMBODIMENT 30

The use of embodiment 29, wherein the woman suffers or suffered from an implantation failure and/or is subject to an IVF treatment.

### BRIEF DESCRIPTION OF THE DRAWINGS

FIG. 1 depicts the endometrial status of a woman in a natural cycle or a hormone replacement therapy cycle. LH+5: five days after an endogenous luteinizing hormone (LH) surge in the woman; LH+7: seven days after an endogenous LH surge in the woman; and LH+9: nine days after an endogenous LH surge in the woman. P+3: three days after a progesterone administration in the woman; P+5: five days after a progesterone administration in the woman; and P+7: seven days after a progesterone administration in the woman.

FIG. 2 depicts a workflow of an endometrial receptivity test, using MIRA PanelChip targeting the 167 miRNAs according to this disclosure.

FIG. 3 depicts processes on how a computer-based algorithm (MIRA Model) is built and how MIRA Model produces a test result.

FIG. 4A shows an exemplary analysis of endometrium receptivity that classifies the endometrial status into one of the three states: a pre-receptive state, a receptive state, or a post-receptive state.

FIG. 4B shows exemplary implantation results in women classified under the three receptive states.

FIG. 5 shows a 10-fold cross-validation and pregnancy rate using miRNA expression profiles comprising expression levels of 167 miRNAs from 183 endometrial samples. SEN: Sensitivity=True Positives/(True Positives+False Negatives); SPE: Specificity=True Negatives/(True Negatives+False Positives); PPV: precision or positive predictive value=True Positives/(True Positives+False Positives); and NPV: negative predictive value=True Negatives/(True Negatives+False Negatives). P+6: embryo implantation six days after a progesterone administration in a woman whose endometrium was previously determined to be in the pre-receptive state; P+5: embryo implantation five days after a progesterone administration in a woman whose endometrium was previously determined to be in the receptive state; and P+4.5: embryo implantation 4.5 days (i.e., 108 hours) after a progesterone administration in a woman whose endometrium was previously determined to be in the post-receptive state.

FIG. 6 shows the MIRA scoring system, classifying the endometrial samples into one of the three states: a pre-receptive state, a receptive state, or a post-receptive state, depending on the value of the receptivity predictive score.

### DESCRIPTION OF THE EMBODIMENTS

The disclosures and embodiments set forth herein are to be construed as exemplary only and not as limiting the scope of the invention. Although specific terms are employed herein, unless otherwise noted, they are used in a generic and descriptive sense only and not for purposes of limitation.

### DEFINITIONS

As used herein, the singular forms “a,” “an,” and “the” are intended to include the plural forms as well, unless the context clearly indicates otherwise.

The term “cDNA” refers to complementary DNA generated by performing reverse transcription on an RNA preparation using a reverse transcriptase. In some embodiments, the RNA preparation contains miRNAs extracted from an endometrial tissue sample. See Example 1.

The terms “comprise,” “have” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and can also cover other unlisted steps. Similarly, any composition or kit that “comprises,” “has” or “includes” one or more features is not limited to possessing only those one or more features and can cover other unlisted features. The use of any and all examples, or exemplary language (e.g., “such as”) provided with respect to certain embodiments herein is intended merely to better illuminate the present disclosure and does not pose a limitation on the scope of the present disclosure otherwise claimed.

The term “expression” refers to the transcription and/or accumulation of RNA molecules in a biological sample, for example, an endometrial tissue sample from a woman. In this context, the term “miRNA expression” refers to the amount of one or more miRNAs in a biological sample, and the miRNA expression can be detected by using suitable methods known in the art. See, e.g., Example 1.

The term “microRNA” or “miRNA” refers to a class of approximately 18 to 25 nucleotide long non-coding RNA derived from an endogenous gene. miRNAs function as post-transcriptional regulators of gene expression by base pairing to the 3′ untranslated regions (UTR) of their target mRNAs for mRNA degradation or translation inhibition.

The terms “nucleic acid,” “nucleotide” and “polynucleotide” are used interchangeably and refer to a polymer of DNA or RNA in either single or double stranded form. Unless otherwise noted, these terms encompass polynucleotides containing known analogues of natural nucleotides that have similar binding properties as the reference nucleic acid and are metabolized in a manner similar to naturally occurring nucleotides.

The term “primer” refers to an oligonucleotide which acts to initiate synthesis of a complementary nucleic acid strand when placed under conditions in which synthesis of a primer extension product is induced, e.g., in the presence of nucleotides and a polymerization-inducing agent such as a DNA or RNA polymerase and at a suitable temperature, pH, metal ion concentration, and salt concentration.

The term “probe” refers to a structure comprising a polynucleotide, which contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with their use in specific detection of all or a portion of a target sequence of a target nucleic acid.

The term “qPCR” or “quantitative PCR” refers to an experimental method of using polymerase chain reaction to amplify and quantify target DNA and/or RNA at the same time. Quantification is performed using a plurality of chemical substances (including, for instance, fluorescent dye of SYBR® green or fluorescent reporter oligonucleotide probe of Taqman probe), and real-time quantification is performed by measuring the amplified DNA and/or RNA in the reaction after one or more amplification cycles.

The term “targeting” refers to the selection of suitable nucleotide sequences that hybridize to a nucleic acid sequence of interest. In some embodiments, the nucleic acid sequence of interest includes a miRNA having the sequence of any one of SEQ ID NOs: 1-167. See Example 1.

### Overview of the Methods for Determining Endometrial Status

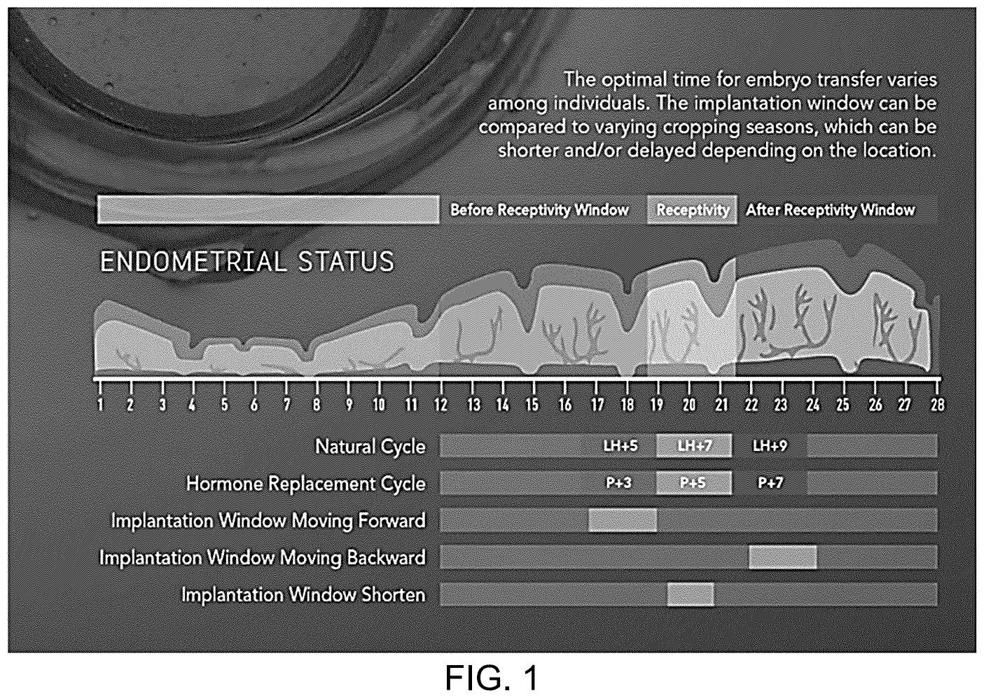

Endometrial receptivity is the state in which a woman's endometrium is prepared for embryo implantation. This occurs in all menstrual cycles in a time period referred to as WOI. As shown in FIG. 1, in a natural cycle, ovulation occurs after the LH surge, and the WOI is around seven days after the LH surge (LH+7). In a hormone replacement therapy cycle, the WOI is around five days after a progesterone administration (P+5). These estimates give probable information on endometrial receptivity. However, the ultimate answer for the endometrium status can only be provided by an examination of the endometrium itself.

To that end, an endometrial sample can be collected from the uterine cavity of a women either five days after a progesterone administration (P+5) in a hormone replacement therapy cycle or seven days after an endogenous LH surge (LH+7) in a natural cycle. The sample is then subject to a molecular diagnostic tool that analyzes the endometrial receptivity status. In the methods of determining an endometrial status according to this disclosure, the molecular diagnostic tool analyzes the miRNA expression profile of the endometrial sample.

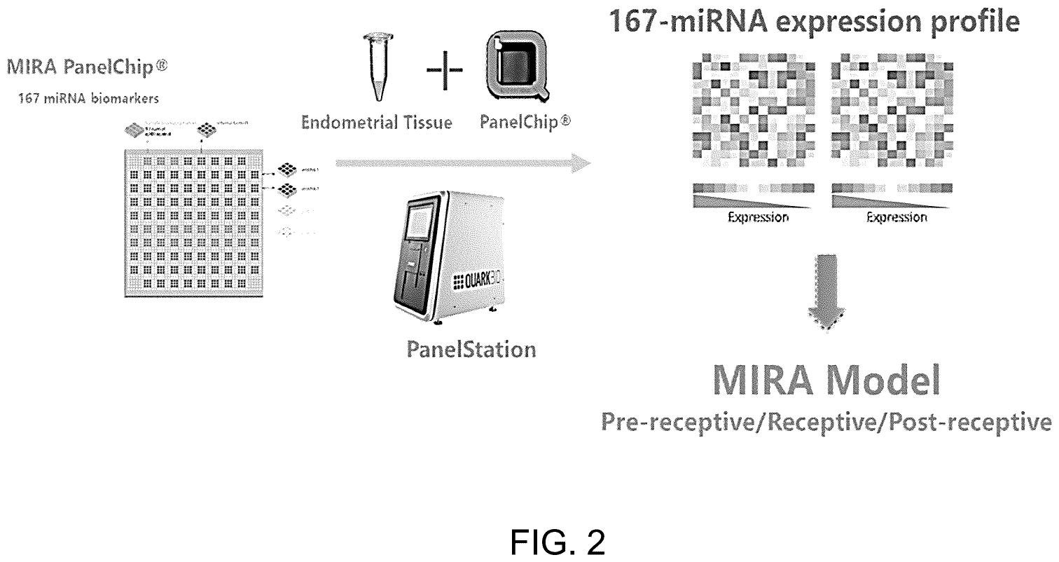

As shown in FIG. 2, the present disclosure provides methods of determining an endometrial status, comprising: (a) performing an assay on an endometrial sample to determine a miRNA expression profile of the endometrial sample, wherein the miRNA expression profile comprises expression levels of a plurality of miRNAs, for example, 167 miRNAs having the sequences of SEQ ID NOs: 1-167, respectively; and (b) analyzing the miRNA expression profile with an a computer-based algorithm to obtain a receptivity predictive score, wherein the receptivity predictive score classifies the endometrial status into a pre-receptive state, a receptive state, or a post-receptive state.

The pre-receptive state indicates that the endometrium is not yet ready to receive the embryo and embryo implantation at this time may be too early. The receptive state (WOI) indicates that the endometrium is at an optimal time for embryo implantation. The post-receptive state indicates that the endometrium already passed the optimal stage for embryo implantation.

### Analyzing miRNA Expression Profile to Determine Endometrial Receptivity

The present disclosure determines an endometrial sample's miRNA expression profile. In some embodiments, the miRNA expression profile comprises expression levels of a plurality of miRNAs, for example, at least 10, 25, 50, 75, 100, 125, 150, or 200 miRNAs, all of which may be implicated in the regulation of endometrial receptivity. In preferred embodiments, the present disclosure provides a selection of 167 miRNAs, whose expression levels have been implicated in the regulation of endometrial receptivity. See Example 1. These 167 miRNAs were chosen by first identifying genes involved in the reproductive diseases from the Human Disease Ontology database, and then selecting potential regulator miRNAs using miRTARBase, TargetScan, and miRDB.

In order to determine an endometrial status, the methods according to this disclosure comprise performing an assay to determine the miRNA expression profile of the endometrial sample, wherein the miRNA expression profile comprises expression levels of the 167 miRNAs shown in Table 1.

TABLE 1
Names and sequences of the 167 miRNAs.

| | | SEQ |
|---|---|---|
| | | ID |
| Name | Sequence | NO |
| hsa-miR-155-5p | UUAAUGCUAAUCGUGAUAGGGGUU | 1 |
| hsa-miR-145-5p | GUCCAGUUUUCCCAGGAAUCCCU | 2 |
| hsa-miR-34a-5p | UGGCAGUGUCUUAGCUGGUUGU | 3 |
| hsa-miR-21-5p | UAGCUUAUCAGACUGAUGUUGA | 4 |
| hsa-miR-125b-5p | UCCCUGAGACCCUAACUUGUGA | 5 |
| hsa-miR-29a-3p | UAGCACCAUCUGAAAUCGGUUA | 6 |
| hsa-miR-29b-3p | UAGCACCAUUUGAAAUCAGUGUU | 7 |
| hsa-miR-200c-3p | UAAUACUGCCGGGUAAUGAUGGA | 8 |
| hsa-miR-24-3p | UGGCUCAGUUCAGCAGGAACAG | 9 |
| hsa-miR-9-5p | UCUUUGGUUAUCUAGCUGUAUGA | 10 |
| hsa-miR-146a-5p | UGAGAACUGAAUUCCAUGGGUU | 11 |
| hsa-miR-26a-5p | UUCAAGUAAUCCAGGAUAGGCU | 12 |
| hsa-miR-17-5p | CAAAGUGCUUACAGUGCAGGUAG | 13 |
| hsa-miR-200b-3p | UAAUACUGCCUGGUAAUGAUGA | 14 |
| hsa-miR-221-3p | AGCUACAUUGUCUGCUGGGUUUC | 15 |
| hsa-miR-181a-5p | AACAUUCAACGCUGUCGGUGAGU | 16 |
| hsa-miR-122-5p | UGGAGUGUGACAAUGGUGUUUG | 17 |
| hsa-miR-199a-5p | CCCAGUGUUCAGACUACCUGUUC | 18 |
| hsa-miR-29c-3p | UAGCACCAUUUGAAAUCGGUUA | 19 |
| hsa-miR-31-5p | AGGCAAGAUGCUGGCAUAGCU | 20 |
| hsa-miR-1-3p | UGGAAUGUAAAGAAGUAUGUAU | 21 |
| hsa-miR-20a-5p | UAAAGUGCUUAUAGUGCAGGUAG | 22 |
| hsa-miR-27a-3p | UUCACAGUGGCUAAGUUCCGC | 23 |
| hsa-miR-203a-3p | GUGAAAUGUUUAGGACCACUAG | 24 |
| hsa-miR-141-3p | UAACACUGUCUGGUAAAGAUGG | 25 |
| hsa-miR-200a-3p | UAACACUGUCUGGUAACGAUGU | 26 |
| hsa-miR-22-3p | AAGCUGCCAGUUGAAGAACUGU | 27 |
| hsa-miR-101-3p | UACAGUACUGUGAUAACUGAA | 28 |
| hsa-miR-16-5p | UAGCAGCACGUAAAUAUUGGCG | 29 |
| hsa-miR-182-5p | UUUGGCAAUGGUAGAACUCACACU | 30 |
| hsa-miR-210-3p | CUGUGCGUGUGACAGCGGCUGA | 31 |
| hsa-miR-125a-5p | UCCCUGAGACCCUUUAACCUGUGA | 32 |
| hsa-let-7a-5p | UGAGGUAGUAGGUUGUAUAGUU | 33 |
| hsa-miR-23a-3p | AUCACAUUGCCAGGGAUUUCC | 34 |
| hsa-miR-19a-3p | UGUGCAAAUCUAUGCAAAACUGA | 35 |
| hsa-miR-223-3p | UGUCAGUUUGUCAAAUACCCCA | 36 |
| hsa-miR-143-3p | UGAGAUGAAGCACUGUAGCUC | 37 |
| hsa-miR-205-5p | UCCUUCAUUCCACCGGAGUCUG | 38 |
| hsa-miR-30a-5p | UGUAAACAUCCUCGACUGGAAG | 39 |
| hsa-miR-133a-3p | UUUGGUCCCCUUCAACCAGCUG | 40 |
| hsa-miR-126-3p | UCGUACCGUGAGUAAUAAUGCG | 41 |
| hsa-miR-128-3p | UCACAGUGAACCGGUCUCUUU | 42 |
| hsa-miR-222-3p | AGCUACAUCUGGCUACUGGGU | 43 |
| hsa-miR-214-3p | ACAGCAGGCACAGACAGGCAGU | 44 |
| hsa-miR-133b | UUUGGUCCCCUUCAACCAGCUA | 45 |
| hsa-miR-181b-5p | AACAUUCAUUGCUGUCGGUGGGU | 46 |
| hsa-miR-15a-5p | UAGCAGCACAUAAUGGUUUGUG | 47 |
| hsa-miR-106a-5p | AAAAGUGCUUACAGUGCAGGUAG | 48 |
| hsa-miR-429 | UAAUACUGUCUGGUAAAACCGU | 49 |
| hsa-miR-7-5p | UGGAAGACUAGUGAUUUUGUUGUU | 50 |
| hsa-miR-106b-5p | UAAAGUGCUGACAGUGCAGAU | 51 |
| hsa-miR-10b-5p | UACCCUGUAGAACCGAAUUUGUG | 52 |
| hsa-miR-192-5p | CUGACCUAUGAAUUGACAGCC | 53 |
| hsa-miR-195-5p | UAGCAGCACAGAAAUAUUGGC | 54 |
| hsa-miR-30c-5p | UGUAAACAUCCUACACUCUCAGC | 55 |
| hsa-miR-335-5p | UCAAGAGCAAUAACGAAAAAUGU | 56 |
| hsa-let-7b-5p | UGAGGUAGUAGGUUGUGUGGUU | 57 |
| hsa-miR-224-5p | UCAAGUCACUAGUGGUUCCGUUUAG | 58 |
| hsa-miR-135a-5p | UAUGGCUUUUUAUUCCUAUGUGA | 59 |
| hsa-miR-206 | UGGAAUGUAAGGAAGUGUGUGG | 60 |
| hsa-miR-92a-3p | UAUUGCACUUGUCCCGGCCUGU | 61 |
| hsa-miR-150-5p | UCUCCCAACCCUUGUACCAGUG | 62 |
| hsa-miR-15b-5p | UAGCAGCACAUCAUGGUUUACA | 63 |
| hsa-miR-130a-3p | CAGUGCAAUGUUAAAAGGGCAU | 64 |
| hsa-miR-130b-3p | CAGUGCAAUGAUGAAAGGGCAU | 65 |
| hsa-miR-140-5p | CAGUGGUUUUACCCUAUGGUAG | 66 |
| hsa-miR-18a-5p | UAAGGUGCAUCUAGUGCAGAUAG | 67 |
| hsa-let-7c-5p | UGAGGUAGUAGGUUGUAUGGUU | 68 |
| hsa-miR-196a-5p | UAGGUAGUUUCAUGUUGUUGGG | 69 |
| hsa-miR-199a-3p | ACAGUAGUCUGCACAUUGGUUA | 70 |
| hsa-miR-103a-3p | AGCAGCAUUGUACAGGGCUAUGA | 71 |
| hsa-miR-129-5p | CUUUUUGCGGUCUGGGCUUGC | 72 |
| hsa-miR-152-3p | UCAGUGCAUGACAGAACUUGG | 73 |
| hsa-miR-144-3p | UACAGUAUAGAUGAUGUACU | 74 |
| hsa-miR-183-5p | UAUGGCACUGGUAGAAUUCACU | 75 |
| hsa-miR-93-5p | CAAAGUGCUGUUCGUGCAGGUAG | 76 |
| hsa-miR-100-5p | AACCCGUAGAUCCGAACUUGUG | 77 |
| hsa-miR-19b-3p | UGUGCAAAUCCAUGCAAAACUGA | 78 |
| hsa-miR-30b-5p | UGUAAACAUCCUACACUCAGCU | 79 |
| hsa-miR-373-3p | GAAGUGCUUCGAUUUUGGGGUGU | 80 |
| hsa-miR-451a | AAACCGUUACCAUUACUGAGUU | 81 |
| hsa-miR-142-3p | UGUAGUGUUUCCUACUUUAUGGA | 82 |
| hsa-miR-20b-5p | CAAAGUGCUCAUAGUGCAGGUAG | 83 |
| hsa-miR-30d-5p | UGUAAACAUCCCCGACUGGAAG | 84 |
| hsa-miR-372-3p | AAAGUGCUGCGACAUUUGAGCGU | 85 |
| hsa-miR-135b-5p | UAUGGCUUUUCAUUCCUAUGUGA | 86 |
| hsa-miR-193a-3p | AACUGGCCUACAAAGUCCCAGU | 87 |
| hsa-miR-409-3p | GAAUGUUGCUCGGUGAACCCCU | 88 |
| hsa-let-7g-5p | UGAGGUAGUAGUUUGUACAGUU | 89 |
| hsa-miR-10a-5p | UACCCUGUAGAUCCGAAUUUGUG | 90 |
| hsa-miR-191-5p | CAACGGAAUCCCAAAAGCAGCUG | 91 |
| hsa-let-7f-5p | UGAGGUAGUAGAUUGUAUAGUU | 92 |
| hsa-miR-134-5p | UGUGACUGGUUGACCAGAGGGG | 93 |
| hsa-miR-146b-5p | UGAGAACUGAAUUCCAUAGGCUG | 94 |
| hsa-miR-127-3p | UCGGAUCCGUCUGAGCUUGGCU | 95 |
| hsa-miR-196b-5p | UAGGUAGUUUCCUGUUGUUGGG | 96 |
| hsa-miR-302d-3p | UAAGUGCUUCCAUGUUUGAGUGU | 97 |
| hsa-miR-663a | AGGCGGGGCGCCGCGGGACCGC | 98 |
| hsa-miR-326 | CCUCUGGGCCCUUCCUCCAG | 99 |
| hsa-miR-486-5p | UCCUGUACUGAGCUGCCCCGAG | 100 |
| hsa-miR-17-3p | ACUGCAGUGAAGGCACUUGUAG | 101 |
| hsa-miR-30e-5p | UGUAAACAUCCUUGACUGGAAG | 102 |
| hsa-let-7d-5p | AGAGGUAGUAGGUUGCAUAGUU | 103 |
| hsa-miR-193b-3p | AACUGGCCCUCAAAGUCCCGCU | 104 |
| hsa-miR-202-3p | AGAGGUAUAGGGCAUGGGAA | 105 |
| hsa-miR-216a-5p | UAAUCUCAGCUGGCAACUGUGA | 106 |
| hsa-miR-376c-3p | AACAUAGAGGAAAUUCCACGU | 107 |
| hsa-miR-198 | GGUCCAGAGGGGAGAUAGGUUC | 108 |
| hsa-miR-215-5p | AUGACCUAUGAAUUGACAGAC | 109 |
| hsa-miR-197-3p | UUCACCACCUUCUCCACCCAGC | 110 |
| hsa-miR-29a-5p | ACUGAUUUCUUUUGGUGUUCAG | 111 |
| hsa-miR-425-5p | AAUGACACGAUCACUCCCGUUGA | 112 |
| hsa-miR-574-3p | CACGCUCAUGCACACACCCACA | 113 |
| hsa-miR-18b-5p | UAAGGUGCAUCUAGUGCAGUUAG | 114 |
| hsa-miR-483-5p | AAGACGGGAGGAAAGAAGGGAG | 115 |
| hsa-miR-625-5p | AGGGGGAAAGUUCUAUAGUCC | 116 |
| hsa-miR-338-5p | AACAAUAUCCUGGUGCUGAGUG | 117 |
| hsa-miR-539-5p | GGAGAAAUUAUCCUUGGUGUGU | 118 |
| hsa-miR-151a-3p | CUAGACUGAAGCUCCUUGAGG | 119 |
| hsa-miR-208b-3p | AUAAGACGAACAAAAGGUUUGU | 120 |
| hsa-miR-330-5p | UCUCUGGGCCUGUGUCUUAGGC | 121 |
| hsa-miR-382-5p | GAAGUUGUUCGUGGUGGAUUCG | 122 |
| hsa-miR-499a-5p | UUAAGACUUGCAGUGAUGUUU | 123 |
| hsa-miR-223-5p | CGUGUAUUUGACAAGCUGAGUU | 124 |
| hsa-miR-31-3p | UGCUAUGCCAACAUAUUGCCAU | 125 |
| hsa-miR-361-5p | UUAUCAGAAUCUCCAGGGGUAC | 126 |
| hsa-miR-423-3p | AGCUCGGUCUGAGGCCCCUCAGU | 127 |
| hsa-miR-885-5p | UCCAUUACACUACCCUGCCUCU | 128 |
| hsa-miR-95-3p | UUCAACGGGUAUUUAUUGAGCA | 129 |
| hsa-miR-99b-5p | CACCCGUAGAACCGACCUUGCG | 130 |
| hsa-miR-299-5p | UGGUUUACCGUCCCACAUACAU | 131 |
| hsa-miR-378a-5p | CUCCUGACUCCAGGUCCUGUGU | 132 |
| hsa-miR-500a-5p | UAAUCCUUGCUACCUGGGUGAGA | 133 |
| hsa-miR-518a-5p | CUGCAAAGGGAAGCCCUUUC | 134 |
| hsa-miR-589-5p | UGAGAACCACGUCUGCUCUGAG | 135 |
| hsa-miR-718 | CUUCCGCCCCGCCGGGCGUCG | 136 |
| hsa-miR-940 | AAGGCAGGGCCCCCGCUCCCC | 137 |
| hsa-miR-28-3p | CACUAGAUUGUGAGCUCCUGGA | 138 |
| hsa-miR-411-5p | UAGUAGACCGUAUAGCGUACG | 139 |
| hsa-miR-423-5p | UGAGGGGCAGAGAGCGAGACUUU | 140 |
| hsa-miR-450a-5p | UUUUGCGAUGUGUUCCUAAUAU | 141 |
| hsa-miR-484 | UCAGGCUCAGUCCCCUCCCGAU | 142 |
| hsa-miR-593-5p | AGGCACCAGCCAGGCAUUGCUCAGC | 143 |
| hsa-miR-652-3p | AAUGGCGCCACUAGGGUUGUG | 144 |
| hsa-miR-760 | CGGCUCUGGGUCUGUGGGGA | 145 |
| hsa-miR-1228-5p | GUGGGCGGGGGCAGGUGUGUG | 146 |
| hsa-miR-1254 | AGCCUGGAAGCUGGAGCCUGCAGU | 147 |
| hsa-miR-1290 | UGGAUUUUUGGAUCAGGGA | 148 |
| hsa-miR-574-5p | UGAGUGUGUGUGUGUGAGUGUGU | 149 |
| hsa-miR-579-3p | UUCAUUUGGUAUAAACCGCGAUU | 150 |
| hsa-miR-596 | AAGCCUGCCCGGCUCCUCGGG | 151 |
| hsa-miR-601 | UGGUCUAGGAUUGUUGGAGGAG | 152 |
| hsa-miR-660-5p | UACCCAUUGCAUAUCGGAGUUG | 153 |
| hsa-let-7d-3p | CUAUACGACCUGCUGCCUUUCU | 154 |
| hsa-miR-1225-3p | UGAGCCCCUGUGCCGCCCCCAG | 155 |
| hsa-miR-1248 | ACCUUCUUGUAUAAGCACUGUGCUAAA | 156 |
| hsa-miR-1972 | UCAGGCCAGGCACAGUGGCUCA | 157 |
| hsa-miR-1973 | ACCGUGCAAAGGUAGCAUA | 158 |
| hsa-miR-2114-3p | CGAGCCUCAAGCAAGGGACUU | 159 |
| hsa-miR-217-5p | UACUGCAUCAGGAACUGAUUGGA | 160 |
| hsa-miR-320a-3p | AAAAGCUGGGUUGAGAGGGCGA | 161 |
| hsa-miR-375-3p | UUUGUUCGUUCGGCUCGCGUGA | 162 |
| hsa-miR-425-3p | AUCGGGAAUGUCGUGUCCGCCC | 163 |
| hsa-miR-4306 | UGGAGAGAAAGGCAGUA | 164 |
| hsa-miR-452-3p | CUCAUCUGCAAAGAAGUAAGUG | 165 |
| hsa-miR-4772-3p | CCUGCAACUUUGCCUGAUCAGA | 166 |
| hsa-miR-520b-3P | AAAGUGCUUCCUUUUAGAGGG | 167 |

The expression levels of the miRNAs can be analyzed with quantitative methods known in the art. In some embodiments, to facilitate the analysis, one or more miRNA profiling chips targeting these 167 miRNAs can be used. For example, in Example 1, two miRNA profiling chips are designed and developed to analyze the expression levels of these 167 miRNAs. In some embodiments, the one or more chips additionally target certain RNA sequences, e.g., 18s rRNA, that can be used as the endogenous controls for the miRNA expression analysis. See Example 1.

The present disclosure provides methods of determining the miRNA expression profile of an endometrial sample. The method generally comprises (i) obtaining or having obtained an endometrial sample from a woman's uterine cavity, (ii) performing an assay to determine a miRNA expression profile of the endometrial sample, wherein the miRNA expression profile comprises expression levels of a plurality of miRNAs, for example, 167 miRNAs having the sequences of SEQ ID NOs: 1-167, respectively.

In some embodiments, the endometrial sample may be obtained via an invasive method, for example, by taking a small biopsy from the endometrium. See Example 1. In some embodiments, the endometrial sample may be obtained via a less invasive method, for example, by collecting the detached cells present in the uterine lavage. Without wishing to be bound by any theory, it is believed that the claimed qPCR-based miRNA expression profiling method provides higher specificity and sensitivity as compared to the microarray-based mRNA expression profiling method, such that a significantly less amount of the endometrial sample may be needed in the methods according to this disclosure. See Wang et al., “Large scale real-time PCR validation on gene expression measurements from two commercial long-oligonucleotide microarrays,” BMC Genomics, 2006, 7:59-75.

In some embodiments, the endometrial sample is obtained seven days after an endogenous LH surge (LH+7) in the woman. In some embodiments, the endometrial sample is obtained five days after a progesterone administration (P+5) in the woman.

The miRNAs in the endometrial sample can be extracted and enriched using methods known in the art. For example, miRNA can be extracted from the endometrial tissue using the miRNeasy Micro Kit (QIAGEN) following the manufacturer's instructions. See Example 1. The miRNA-enriched preparations can be stored at −80°° C. The quantity and quality of the miRNA can be analyzed using methods known in the art. For example, the miRNA can be analyzed using a commercially available Agilent bioanalyzer.

The expression level of each miRNA can be quantified by methods known in the art, including qPCR, sequencing, microarray, or RNA-DNA hybrid capture technology. In some embodiments, the methods according to this disclosure use a qPCR reaction, which generally has higher sensitivity and specificity than northern blot hybridization and/or microarray gene chip analysis. To that end, cDNA can be synthesised from the extracted and enriched miRNAs in a reverse transcription reaction, and a qPCR reaction can be performed to quantify the expression levels of the miRNAs. Thus, in some embodiments, the miRNA expression profile is determined by qPCR, optionally using one or more miRNA profiling chips disclosed herein. See Example 1.

Currently, qPCR assays can be divided into two types. The first type is performing cDNA synthesis using a stem-loop reverse transcription primer, and quantifying miRNA using a miRNA specific probe or a universal probe. The second method is to perform cDNA synthesis using a linear universal reverse transcription primer and quantify miRNA using a miRNA specific forward primer, a reverse transcription-primer specific reverse primer, and a double-stranded DNA intercalating dye.

In some embodiments, the cDNA synthesis is performed using a universal reverse transcription primer as disclosed in U.S. Pat. No. 10,590,478, which is incorporated herein by reference. In some embodiments, the cDNA synthesis is performed using a universal reverse transcription primer having a nucleotide sequence represented by the following general formula: 5′-R-(dT)nVN-3′, wherein R comprises the sequence of CAACTCAGGTCGTAGGCAATTCGT (SEQ ID NO:168), (dT)n is an n number of continuous thymine residues, wherein n is 19, V is an adenine residue, a guanine residue, or a cytosine residue, and N is an adenine residue, a guanine residue, a cytosine residue, or a thymine residue.

To reduce cost and for ease of use, in some embodiments, the qPCR reactions can be performed using one or more miRNA profiling chips that target all of the 167 miRNAs according to this disclosure. See Example 1. In some embodiments, each of the miRNA profiling chips is preloaded with suitable primers and/or probes capable of concurrently analyzing the expression of at least 20, 30, 40, 50, 60, 60, 70, 80, 90, 95, 96, 97, 98, 99, 100, 110, 120, 130, 140, 150, 160, 170, 180, 190, or 200 miRNAs. In some embodiments, the miRNA profiling chip contains a multiplex slide plate as disclosed in U.S. Pat. No. 9,724,692, U.S. Pat. No. 10,415,084, application Ser. No. 16/191,451 and application Ser. No. 16/233,121 which are incorporated herein by reference.

The qPCR reactions can be performed using methods known in the art. In some embodiments, the qPCR reactions can be carried out using a thermal cycler device as disclosed in U.S. Pat. No. 9,168,533 and application Ser. No. 16/559,642, which are incorporated herein by reference. See also Example 1.

### miRNA Analysis Algorithm and its use for Determining Endometrial Receptivity

According to the methods of this disclosure, the miRNA expression profile can be used to generate a receptivity predictive score, using a computer-based miRNA analysis algorithm. The receptivity predictive score classifies the endometrial status into one of the following three states: a pre-receptive state, a receptive state, or a post-receptive state.

The computer-based miRNA analysis algorithm is a mathematical prediction classifier which uses the miRNA expression data and learns to distinguish classes according to different receptivity states.

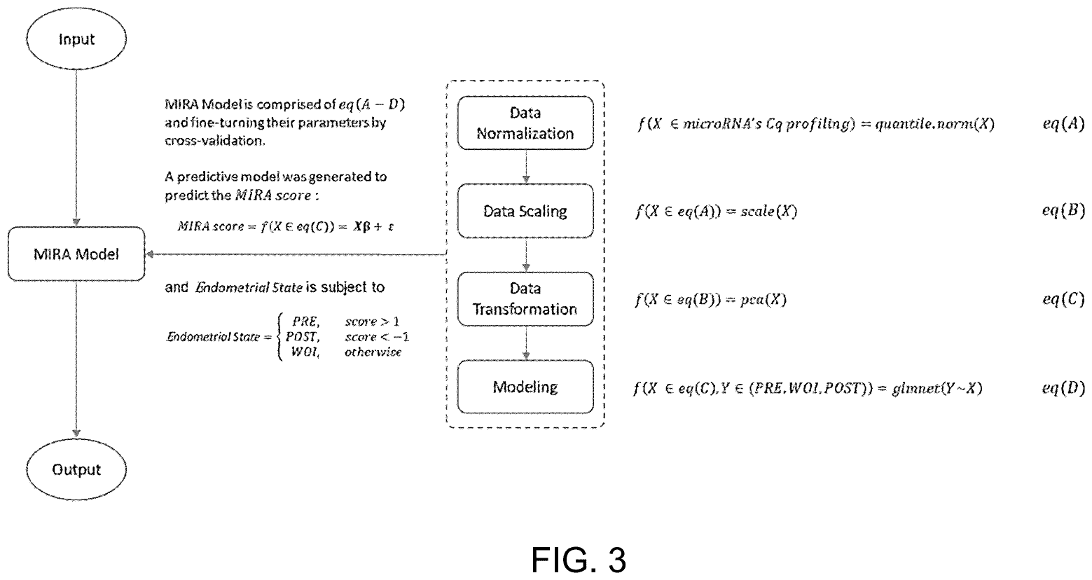

To build the algorithm, the raw data on miRNA expression levels is divided into a training set and a validation set. The training set is used to train the prediction classifier and the validation set is used to evaluate and refine the performance of the prediction classifier. As shown in FIG. 3, one or more of the following steps are performed to build and validate the algorithm: data normalization, data scaling, data transformation, prediction modeling, and cross-validation.

In order to make distributions identical in statistical properties, the data can be normalized by Quantile Normalization, as described in Bolstad et al., “A comparison of normalization methods for high density oligonucleotide array data based on variance and bias,” Bioinformatics, 2003, 19(2): 185-193. Furthermore, to ensure that the objective functions are working properly, the data can be standardized the range of value to make data having zero-mean and unit-variance.

For both reasons of data reduction and feature extraction, a principal component analysis (PCA) can be used to condense the information from a large number of original variables and generate a small set of new features by linearly combining the original variables.

The PCA-transformed data can be used to further build a generalized linear model with elastic net regularization, which is a regularized regression method that linearly combined the L1 and L2 penalties of lasso and ridge methods, as described in Zou et al., “Regularization and variable selection via the elastic net,” J. R. Statist. Soc. B, 2005, 67, part 2, 301-320. Additional information on glmnet is known and available at glmnet.stanford.edu.

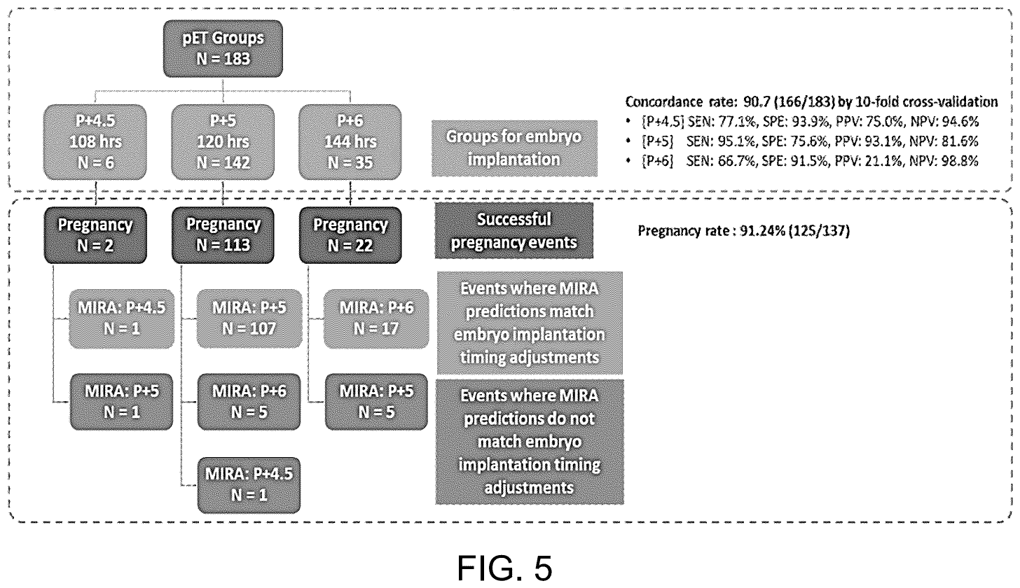

The k-fold cross-validation method, for example, a 10-fold cross-validation, can be used to assess the computer-based miRNA analysis algorithm's predictive value before finalizing it. See FIG. 5. In a k-fold cross-validation, the original sample is randomly partitioned into k equal size subsamples. Of the k subsamples, a single subsample is retained as the validation data for testing the model, and the remaining k−1 subsamples are used as training data. The cross-validation process is then repeated k times (the folds), with each of the k subsamples used exactly once as the validation data. The k results from the folds can then be averaged (or otherwise combined) to produce a single estimation.

Pregnancy rates can be used to assess the predictive value of the computer-based miRNA analysis algorithm. See Example 2.

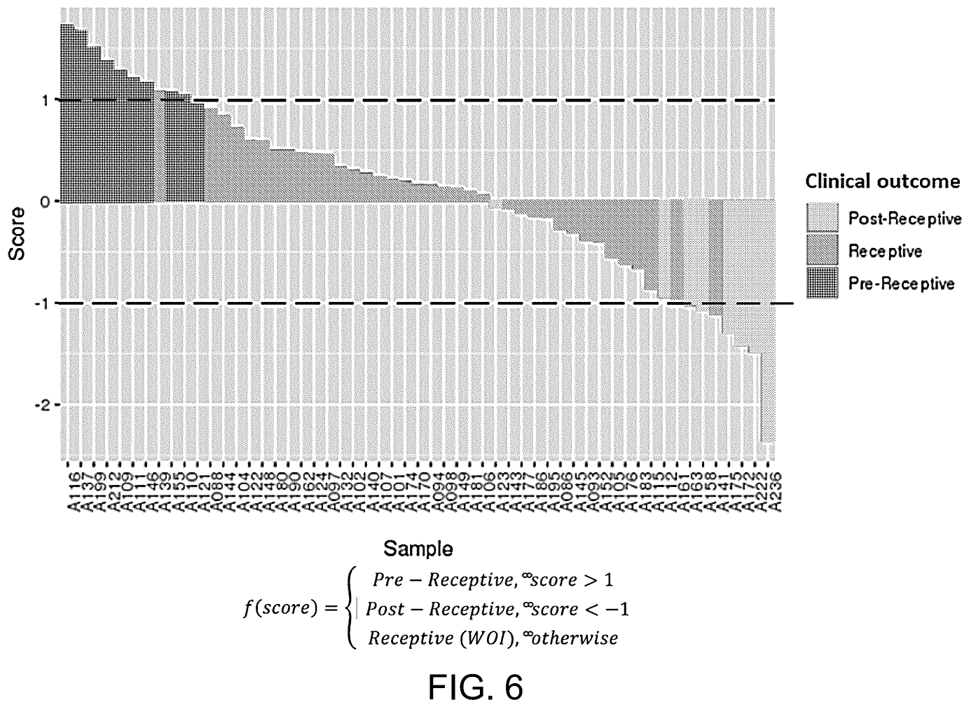

After validation and refinement, a computer-based miRNA analysis algorithm is generated. Running the algorithm generates a receptivity predictive score that classifies the endometrial status of the woman into one of the three states as follows: if the score is greater than 1, the woman's endometrium is in the pre-receptive state; if the score is less than −1, the woman's endometrium is in the post-receptive state; and if the score is from −1 to 1, the woman's endometrium is in the receptive state. See FIG. 6.

### Applications of the Methods According to this Disclosure

The present disclosure provides methods for determining an endometrial status, using a sample, for example, an endometrial biopsy, comprising: (a) performing an assay on the endometrial sample from a woman to determine a miRNA expression profile of the endometrial sample, wherein the miRNA expression profile comprises expression levels of a plurality of miRNAs, for example, 167 miRNAs having the sequences of SEQ ID NOs: 1-167, respectively; and (b) analyzing the miRNA expression profile to obtain a receptivity predictive score using, for example, a computer-based algorithm.

Methods of the present disclosure can be used for various diagnostic and therapeutic purposes, including but not limited to IVF treatment. For example, in some embodiments, based on the endometrial results, the methods may further include implanting an embryo in the woman or administering one or more treatments to the woman who suffers or suffered from an implantation failure. In some embodiments, the present disclosure provides methods of detecting endometrial receptivity for embryo implantation, comprising: (a) performing an assay on an endometrial sample from a woman to determine a miRNA expression profile of the endometrial sample, wherein the miRNA expression profile comprises expression levels of a plurality of miRNAs, for example, 167 miRNAs having the sequences of SEQ ID NOs: 1-167, (b) analyzing the miRNA expression profile to obtain a receptivity predictive score, wherein the receptivity predictive score determines whether the woman has endometrial receptivity, and (c) transferring an embryo to the endometrium of the woman determined to have endometrial receptivity.

In some embodiments, the methods of determining an endometrial status can be used to determine the timing of embryo implantation in a woman. In some embodiments, if the endometrial status is at the receptive state, the woman is considered suitable for embryo implantation. If the endometrial status is at the pre-receptive or the post-receptive state, the woman is considered not suitable for embryo implantation. In some embodiments, when the endometrial status is determined to be at the pre-receptive state or the post-receptive state, the present disclosure provides methods for embryo implantation based on the information on the endometrial status. For example, if the endometrial status is determined to be at the pre-receptive state, during the next cycle, embryo implantation can be performed between 5.5 and 7.5 days, for example, 5.5, 6, 6.5, 7, or 7.5 days after a progesterone administration. Alternatively, if the endometrial status is determined to be at the post-receptive state, during the next cycle, embryo implantation can be performed between 2.5 and 4.5 days, for example, 2.5, 3, 3.5, 4, or 4.5 days after a progesterone administration.

In cases where the endometrium shows a non-receptive state at the time of the sampling, the information gained is instructive, such that the method can be repeated by taking an endometrial sample at another time, modified in line with the results of the first determination. By way of example, if the endometrial status is at the pre-receptive state, the next time point of taking the endometrial sample can be more than seven days after an endogenous LH surge or more than five days after a progesterone administration. For example, the next point of taking the endometrial sample can be between 7.5 and 10.5 days, for example, 7.5, 8, 8.5, 9, 9.5, 10, or 10.5 days after an endogenous LH surge or between 5.5 and 7.5 days, for example, 5.5, 6, 6.5, 7, or 7.5 days after a progesterone administration. Alternatively, if the endometrial status is at the post-receptive state, the next time point of taking the endometrial sample can be fewer than seven days after an endogenous LH surge or fewer than five days after a progesterone administration. For example, the next point of taking the endometrial sample can be between 3.5 and 6.5 days, for example, 3.5, 4, 4.5, 5, 5.5, 6, or 6.5 days after an endogenous LH surge or between 2.5 and 4.5 days, for example, 2.5, 3, 3.5, 4, or 4.5 days after a progesterone administration. By following these procedures, a receptive state can be found, and the success rate of the IVF treatment can be improved. For any one of these uses, the woman suffers or suffered from an implantation failure. In some embodiments, the woman is subject to an IVF treatment.

In some embodiments, if the endometrial status is determined to be at the pre-receptive state or the post-receptive state, the method of determining an endometrial status can be repeated at least once or until the endometrial status is determined to be at the receptive state.

In some embodiments, the methods of determining an endometrial status according to this disclosure can be used to determine the WOI of a woman. In some embodiments, the methods according to this disclosure can be used to classify a woman's responsiveness to the IVF treatment. For any one of these uses, in some embodiments, the woman suffers or suffered from an implantation failure. In some embodiments, the woman is subject to an IVF treatment.

In some embodiments, the methods of determining an endometrial status according to this disclosure can be used as a valuable tool for investigating the effects of pregnancy drugs on the endometrium of a woman. In these embodiments, the woman suffers or suffered from an implantation failure. In some embodiments, the woman is subject to an IVF treatment. Kits

Another aspect of this disclosure relates to kits for carrying out the methods of determining an endometrial status. In some embodiments, the kits comprise primers and/or probes suitable for the detection of the expression levels of a plurality of miRNAs, for example, the 167 miRNAs having the sequences of SEQ ID NOs: 1-167, respectively. See Example 1. In some embodiments, the primers and/or probes are suitable for performing qPCR reactions to detect the expression levels of the 167 miRNAs. In some embodiments, the kits comprise one or more miRNA profiling chips targeting the 167 miRNAs. In some embodiments, the one or more chips additionally target RNA sequences, e.g., 18s rRNA, that can be used as the endogenous controls for the miRNA expression analysis.

The kits may additionally contain instructions on (i) determining a miRNA expression profile of an endometrial sample from a woman, optionally using the one or more miRNA profiling chips, and/or (ii) obtaining a receptivity predictive score based on the miRNA expression profile, using a computer-based algorithm. In some embodiments, the kits contain instructions on how to interpret and use the receptivity predictive score.

In some embodiments, the kits are useful for diagnostic and therapeutic purposes, including but not limited to IVF treatment.

### EXAMPLES

### Example 1

### Materials and methods for Generating a miRNA Expression Profile

Endometrial biopsy. An endometrial biopsy was collected from the uterine cavity of a women using Pipelle Endometrial Suction Curette (Cooper Surgical, Inc.) either five days after a progesterone administration (P+5) in a hormone replacement therapy cycle or seven days after an endogenous luteinizing hormone surge (LH+7) in a natural cycle. Endometrial tissues were stored in RNAlater immediately.

RNA extraction and miRNA enrichment. Total RNA was isolated from the endometrial tissue using the miRNeasy Micro Kit (QIAGEN) following the manufacturer's instructions. Briefly, five mg of the endometrial tissue was disrupted and homogenized in liquid nitrogen with a motor and pestle. 700 μof QIAzol Lysis Reagent was added to the homogenized tissue and the resulting sample was incubated at room temperature for five min to promote the dissociation of nucleoprotein complexes. 140 μl of chloroform per 700 μl of QIAzol Lysis Reagent was added to the tube, and the tube was shaken vigorously by hand for 15 seconds and incubated at room temperature for 2-3 min. The sample was centrifuged at 12,000 g for 15 min at 4° C. After the centrifugation, the upper aqueous phase was transferred to a new tube, one volume of 70% ethanol was added to the tube, and the tube was vortexed thoroughly. The sample was transferred into a RNeasy MinElute spin column and centrifuged at 8,000 g for 15 s at room temperature. The flow-through was pipetted into a 2 ml tube, 0.65 volume of 100% ethanol was added to the flow-through, and the resulting sample was vortexed thoroughly. The sample was then transferred into a RNeasy MinElute spin column and centrifuged at 8,000 g for 15 s at room temperature. The flow-through was discarded, 700 μl Buffer RWT was added to the RNeasy MinElute spin column, and the column was centrifuged for 15 s at 8000 g to wash the column. The flow-through was discarded, 500 μl Buffer RPE was added into the RNeasy MinElute spin column, and the column was centrifuged for 15 s at 8,000 g to wash the column. The flow-through was discarded, 500 μl of 80% ethanol was added to the RNeasy MinElute spin column, and the column was centrifuged for 2 min at 8,000 g to dry the spin column membrane. The RNeasy MinElute spin column was placed into a new 2 ml collection tube and centrifuged for 5 min at 8,000 g. The RNeasy MinElute spin column was placed into a 1.5 ml collection tube, 14-20 μl nuclease-free water was added onto the spin column membrane, and the column was centrifuged for 1 min at 8,000 g to elute the miRNA-enriched fraction. The miRNA-enriched fraction was stored at −80° C.

cDNA synthesis. ≥2 ng of miRNA-enriched fraction from endometrial tissue was used to synthesize cDNA in a 20 μl reverse transcription reaction. Reverse transcription was performed using the QuarkBio microRNA Universal RT Kit (Quark Biosciences Taiwan, Inc.) following the manufacturer's instructions. Briefly, poly-A tails were added to the miRNA using poly-A polymerase, followed by cDNA synthesis. cDNA synthesis was subsequently performed using the following program: 42° C. for 60 min and 95° C. for 5 min, and then 4° C. until completion of program. The synthesized cDNA was stored at −20° C.

miRNA Expression Profiling Using the NextAmp Analysis System and the MIRA PanelChip set. The MIRA PanelChip set contains a total of 167 miRNA assays. The sequences for the 167 miRNAs are shown in Table 1. In addition, RNU6B, RNU43, and 18s rRNA were used as endogenous controls. Three exogenous spike-in controls were used to monitor miRNA extraction, cDNA synthesis, and qPCR efficiency (Quark Biosciences Taiwan, Inc.). The cDNA was analyzed with the MIRA PanelChip set. cDNA (equivalent to 0.1 ng of miRNA-enriched fraction) was added to the mixture containing 30 μl of 2X SYBR Master Mix (Quark Biosciences Taiwan, Inc.), and nuclease-free water was added to the mixture to obtain a final volume of 60 μl. The mixture was mixed by hand thoroughly and briefly spun down to collect the liquid at the bottom. 60 μl of the mixture was dispensed using a Pipetman along the edge of the chip and the mixture was then applied across the entire surface of the MIRA PanelChip via a scraping motion with a glass slide. Each chip was then submerged into a tray containing Channeling Solution (Quark Biosciences Taiwan, Inc.), with reaction wells facing the bottom of the tray. Each tray was then placed into Q Station, which is a thermocycler (see PanelStation in FIG. 2) for MIRA PanelChip applications and includes built-in sample management database and a analysis platform, such that the MIRA PanelChip assays and data analysis can be performed conveniently and quickly. The MIRA PanelChip analysis was subsequently performed according to the following program: 95° C. for 36 s and 60° C. for 72 s, for 40 cycles.

### Example 2

### Computer-based miRNA Analysis Algorithm and its use

As shown in FIG. 3, the computer-based miRNA analysis algorithm (MIRA) was built by performing one or more of the following steps: data normalization, data scaling, data transformation, prediction modeling, and cross-validation.

Data normalization. For making distributions identical in statistical properties, the data was normalized by Quantile Normalization. See equation (A) in FIG. 3; see also Bolstad et al., “A comparison of normalization methods for high density oligonucleotide array data based on variance and bias,” Bioinformatics, 2003, 19(2): 185-193.

Data scaling. To ensure that the objective functions are working properly, the data was standardized the range of value to make data having zero-mean and unit-variance. See equation (B) in FIG. 3.

Data transformation. For the reasons of data reduction and feature extraction, the PCA condensed the information from a large number of original variables and generated a small set of new features by linearly combining the original variables. See equation (C) in FIG. 3.

Modeling. The PCA-transformed data was used to further build a generalized linear model with elastic net regularization, which was a regularized regression method that linearly combined the L1 and L2 penalties of lasso and ridge methods. See equation (D) in FIG. 3; see also Zou et al., “Regularization and variable selection via the elastic net,” J. R. Statist. Soc. B, 2005, 67, part 2, 301-320.

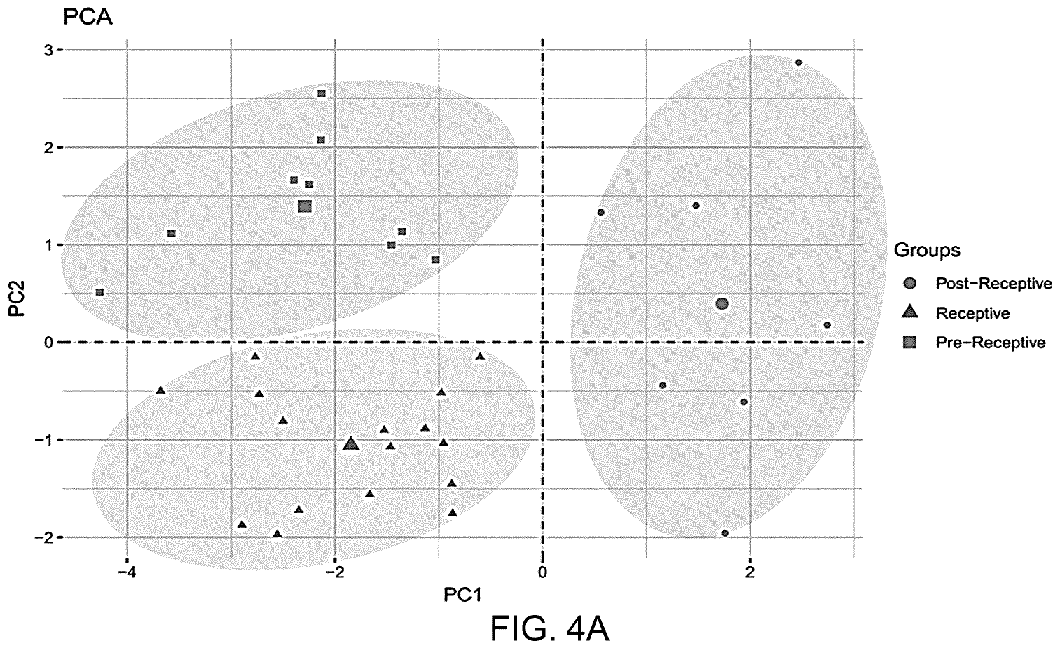

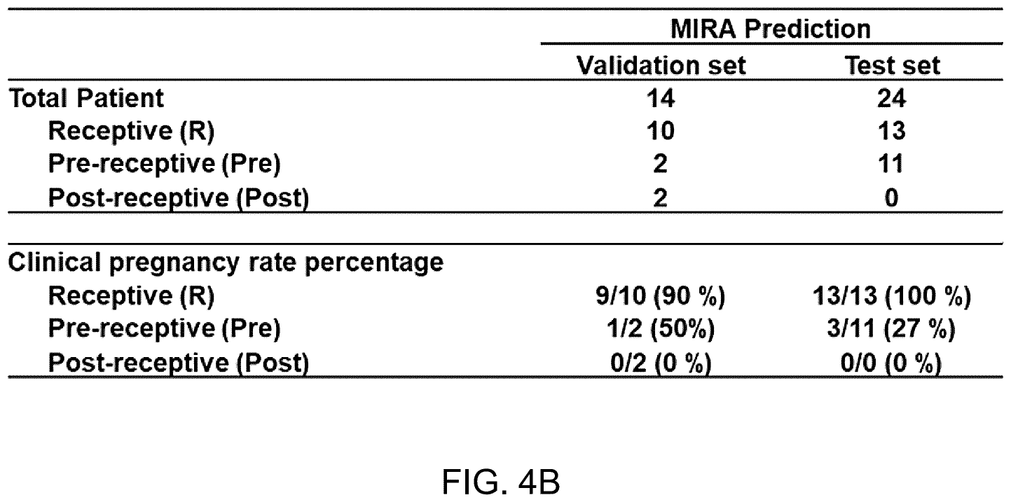

Cross-validations were performed to assess the computer-based miRNA analysis algorithm's predictive value before finalizing the MIRA model. As shown in FIG. 4A, using the miRNA expression profile containing the expression levels of 167 miRNAs having the sequences of SEQ ID NOs: 1-167 shown in Table 1, the MIRA model was able to successfully classify the clinal samples into one of the three status groups: a pre-receptive state, a receptive state, or a post-receptive state. Furthermore, as shown in FIG. 4B, preliminary validation showed a 100% pregnancy rate in women classified under the receptive state (Test set).

Data from 183 women were divided into 10 subsets to achieve the 10-fold cross-validation for model assessment. FIG. 5 shows a 10-fold cross-validation and pregnancy rate using miRNA expression profiles comprising expression levels of 167 miRNAs from 183 endometrial samples. In these tests, in the first cycle, each woman's endometrial status was determined. If a woman's endometrium was determined to be in the pre-receptive state, embryo implantation was performed six days after a progesterone administration in the next cycle (P+6 group; 35 women). If a woman's endometrium was determined to be in the receptive state, embryo implantation was performed five days after a progesterone administration in the next cycle (P+5 group; 142 women). If a woman's endometrium was determined to be in the post-receptive state, embryo implantation was performed 4.5 days after a progesterone administration in the next cycle (P+4.5 group; 6 women). In addition, FIG. 5 shows the sensitivity, specificity, PPV, NPV, and overall concordance rate of the 10-fold cross-validation results.

Among the three groups, 137 pregnancy events were detected, with 22 events from the P+6 group, 113 events from the P+5 group, and 2 events from the P+4.5 group. See FIG. 5, With respect to the predictive evaluation of the computer-based miRNA analysis algorithm, among all 137 pregnancy events, 1 out of 2 from the P+4.5 group, 107 out of 113 from the P+5 group, and 17 out of 22 from the P+6 group showed correct embryo implantation timing adjustments determined by the algorithm and resulted in a 91.24% pregnancy rate (125/137). See FIG. 5.

MIRA Model. Taking into account all of the parameters described in this example (see FIG. 3, eq(A-D) and subsequent fine-tuning their parameters based on cross-validation), a prediction model was generated which classifies all samples into three distinct endometrial states. Running MIRA generated a receptivity predictive score (MIRA score), calculated using the following equation: MIRA score=f(X∈eq(C))=Xβ+ϵ, where β is a vector of coefficients, and ϵ is an error, both being produced by glmnet through the cross-validation (FIG. 3). This model could be applied to any qPCR profiling of an endometrium to predict the endometrial status.

As shown in FIG. 6, running the computer-based miRNA analysis algorithm generated a receptivity predictive score that classifies the endometrial status of the woman into one of the three states: if the score is greater than 1, the woman's endometrium is in the pre-receptive state; if the score is less than −1, the woman's endometrium is in the post-receptive state; and if the score is from −1 to 1, the woman's endometrium is in the receptive state (WOI).

While the disclosure has been particularly shown and described with reference to specific embodiments, it should be understood by those having skill in the art that various changes in form and detail may be made therein without departing from the spirit and scope of the present disclosure.

### REFERENCES

ADDIN EN.REFLIST Ambros, “microRNAs: tiny regulators with great potential,” Cell, 2001, 107(7):823-826.

Bartel, “MicroRNAs: genomics, biogenesis, mechanism, and function,” Cell, 2004, 116(2): 81-97.

Lee et al., “The C. elegans heterochronic gene lin-4 encodes small RNAs with antisense complementarity to lin-14,” Cell, 1993, 75(5):843-854.

Pasquinelli et al., “Conservation of the sequence and temporal expression of let-7 heterochronic regulatory RNA,” Nature, 2000, 408(6808):86-89.

Wightman et al., “Posttranscriptional regulation of the heterochronic gene lin-14 by lin-4 mediates temporal pattern formation in C. elegans,” Cell, 1993, 75(5):855-862.

Navot et al., “The window of embryo transfer and the efficiency of human conception in vitro,” Fertil Steril, 1991, 55(1):114-118.

Harper, “The implantation window,” Baillieres Clin Obstet Gynaecol, 1992, 6(2):351-371.

Hull et al., “Tissue and circulating microRNA influence reproductive function in endometrial disease,” Reprod Biomed Online, 2013, 27(5):515-529.

Galliano et al., “MicroRNA and implantation,” Fertil Steril, 2014, 101(6): 1531-1544.

Liu et al., “MicroRNA and Embryo Implantation,” Am J Reprod Immunol, 2016, 75(3):263-271.

Shi et al., “Endometrial MicroRNA Signature during the Window of Implantation Changed in Patients with Repeated Implantation Failure,” Chin Med J (Engl), 2017, 130(5):566-573.

Kresowik et al., “MicroRNA-31 is significantly elevated in both human endometrium and serum during the window of implantation: a potential biomarker for optimum receptivity,” Biol Reprod, 2014, 91(1):17.

Li et al., “MicroRNA-30a-3p regulates epithelial-mesenchymal transition to affect embryo implantation by targeting Snai2dagger,” Biol Reprod, 2019, 100(5):1171-1179.

Liang et al., “Role of microRNAs in embryo implantation,” Reprod Biol Endocrinol, 2017, 15(1):90.

Paul et al., “The role of microRNAs in human embryo implantation: a review,” J Assist Reprod Genet, 2019, 36(2):179-187.

Vilella et al., “Hsa-miR-30d, secreted by the human endometrium, is taken up by the pre-implantation embryo and might modify its transcriptome,” Development, 2015, 142(18):3210-3221.

Zheng et al., “MicroRNA-200c impairs uterine receptivity formation by targeting FUT4 and α1,3-fucosylation,” Cell Death Differ, 2017, 24(12):2161-2172.

Bolstad et al., “A comparison of normalization methods for high density oligonucleotide array data based on variance and bias,” Bioinformatics, 2003, 19(2): 185-193.

Zou et al., “Regularization and variable selection via the elastic net,” J. R. Statist. Soc. B, 2005, 67, part 2, 301-320.

Schriml et al., “Human Disease Ontology 2018 update: classification, content and workflow expansion,” Nucleic Acids Res, 2019, 47:D955-D62.

Huang et al., “miRTarBase 2020: updates to the experimentally validated microRNA-target interaction database,”' Nucleic Acids Res, 2020, 48: D148-D54.

Agarwal et al., “Predicting effective microRNA target sites in mammalian mRNAs,” 2015, Elife, 4.

Chen et al., “miRDB: an online database for prediction of functional microRNA targets.” Nucleic Acids Res. 48:D127-D31.

Wang et al., “Large scale real-time PCR validation on gene expression measurements from two commercial long-oligonucleotide microarrays,” BMC Genomics. 2006, 7:59-75.

What is claimed is:

1. A kit comprising: (a) one or more microRNA (miRNA) profiling chips targeting a plurality of miRNAs, and (b) instructions on (i) determining a miRNA expression profile of an endometrial sample from a woman, using the one or more miRNA profiling chips, and (ii) obtaining a receptivity predictive score based on the miRNA expression profile, using a computer-based algorithm, wherein the plurality of miRNAs comprise at least 167 miRNAs having the sequences of SEQ ID NOs: 1-167, respectively.

2. The kit of claim 1, wherein the one or more miRNA profiling chips comprise primers for detection of expression levels of the plurality of miRNAs.

3. The kit of claim 2, wherein the miRNA profiling chips are suitable for performing a quantitative PCR (qPCR), sequencing, microarray, or RNA-DNA hybrid capture assay, to detect the expression levels of the plurality of miRNAs.

4. A method for embryo implantation, comprising:
(i) transferring the embryo to the endometrium of a woman whose endometrial status is at a receptive state;
(ii) transferring the embryo to the endometrium of a woman whose endometrial status is at a pre-receptive state, wherein the embryo is transferred 5.5 to 7.5 days after a progesterone administration to the woman; or
(iii) transferring the embryo to the endometrium of a woman whose endometrial status is at a post-receptive state, wherein the embryo is transferred 2.5 to 4.5 days after a progesterone administration to the woman;
wherein the woman's endometrial status is determined by:
(a) performing or having performed an assay on an endometrial sample from the woman to determine a microRNA (miRNA) expression profile of the endometrial sample, wherein the miRNA expression profile comprises expression levels of a plurality of miRNAs comprising at least 167 miRNAs having the sequences of SEQ ID NOs: 1-167, respectively; and
(b) analyzing or having analyzed the miRNA expression profile to obtain a receptivity predictive score, wherein the receptivity predictive score classifies the woman's endometrial status into the pre-receptive state, the receptive state, or the post-receptive state.

5. The method of claim 4, wherein the endometrial sample is obtained from the uterine cavity of the woman.

6. The method of claim 4, wherein the endometrial sample comprises an endometrial biopsy, an endometrial lavage, or combination thereof.

7. The method of claim 4, wherein the endometrial sample is obtained (i) seven days after an endogenous luteinizing hormone (LH) surge in the woman or (ii) five days after a progesterone administration in the woman.

8. The method of claim 4, wherein the miRNA expression profile is determined by quantitative PCR (qPCR), sequencing, microarray, or RNA-DNA hybrid capture technology.

9. The method of claim 8, wherein the miRNA expression profile is determined by qPCR performed on a cDNA preparation synthesized from the miRNAs in the endometrial sample.

10. The method of claim 9, wherein the cDNA synthesis is performed using a universal reverse transcription primer having a nucleotide sequence represented by the following general formula: 5′-R-(dT)nVN-3′, wherein R comprises SEQ ID NO:168, (dT)n is an n number of continuous thymine residues, n is 19, V is an adenine residue, a guanine residue, or a cytosine residue, and N is an adenine residue, a guanine residue, a cytosine residue, or a thymine residue.

11. The method of claim 4, wherein the receptivity predictive score is a value produced by a computer-based algorithm and calculated using the equation of MIRA score=f(X∈eq(C))=Xβ+ϵ, β being a vector of coefficients, and & being an error.

12. The method of claim 11, where the computer-based algorithm is established by performing one or more of the following steps: data normalization, data scaling, data transformation, prediction modeling, and cross-validation.

13. The method of claim 11, wherein a receptivity predictive score greater than 1 indicates the pre-receptive state, a receptivity predictive score less than −1 indicates the post-receptive state, and a receptivity predictive score from −1 to 1 indicates the receptive state.

14. The method of claim 4, wherein the woman suffers or suffered from an implantation failure.


SEQUENCE LISTING (168 sequences)

SEQ ID NO: 1 - RNA, 24 nt, Homo sapiens
  misc_feature 1..24: note = hsa-miR-155-5p
  source 1..24: mol_type = genomic RNA
ttaatgctaatcgtgataggggtt

SEQ ID NO: 2 - RNA, 23 nt, Homo sapiens
  misc_feature 1..23: note = hsa-miR-145-5p
  source 1..23: mol_type = genomic RNA
gtccagttttcccaggaatccct

SEQ ID NO: 3 - RNA, 22 nt, Homo sapiens
  misc_feature 1..22: note = hsa-miR-34a-5p
  source 1..22: mol_type = genomic RNA
tggcagtgtcttagctggttgt

SEQ ID NO: 4 - RNA, 22 nt, Homo sapiens
  misc_feature 1..22: note = hsa-miR-21-5p
  source 1..22: mol_type = genomic RNA
tagcttatcagactgatgttga

SEQ ID NO: 5 - RNA, 22 nt, Homo sapiens
  misc_feature 1..22: note = hsa-miR-125b-5p
  source 1..22: mol_type = genomic RNA
tccctgagaccctaacttgtga

SEQ ID NO: 6 - RNA, 22 nt, Homo sapiens
  misc_feature 1..22: note = hsa-miR-29a-3p
  source 1..22: mol_type = genomic RNA
tagcaccatctgaaatcggtta

SEQ ID NO: 7 - RNA, 23 nt, Homo sapiens
  misc_feature 1..23: note = hsa-miR-29b-3p
  source 1..23: mol_type = genomic RNA
tagcaccatttgaaatcagtgtt

SEQ ID NO: 8 - RNA, 23 nt, Homo sapiens
  misc_feature 1..23: note = hsa-miR-200c-3p
  source 1..23: mol_type = genomic RNA
taatactgccgggtaatgatgga

SEQ ID NO: 9 - RNA, 22 nt, Homo sapiens
  misc_feature 1..22: note = hsa-miR-24-3p
  source 1..22: mol_type = genomic RNA
tggctcagttcagcaggaacag

SEQ ID NO: 10 - RNA, 23 nt, Homo sapiens
  misc_feature 1..23: note = hsa-miR-9-5p
  source 1..23: mol_type = genomic RNA
tctttggttatctagctgtatga

SEQ ID NO: 11 - RNA, 22 nt, Homo sapiens
  misc_feature 1..22: note = hsa-miR-146a-5p
  source 1..22: mol_type = genomic RNA
tgagaactgaattccatgggtt

SEQ ID NO: 12 - RNA, 22 nt, Homo sapiens
  misc_feature 1..22: note = hsa-miR-26a-5p
  source 1..22: mol_type = genomic RNA
ttcaagtaatccaggataggct

SEQ ID NO: 13 - RNA, 23 nt, Homo sapiens
  misc_feature 1..23: note = hsa-miR-17-5p
  source 1..23: mol_type = genomic RNA
caaagtgcttacagtgcaggtag

SEQ ID NO: 14 - RNA, 22 nt, Homo sapiens
  misc_feature 1..22: note = hsa-miR-200b-3p
  source 1..22: mol_type = genomic RNA
taatactgcctggtaatgatga

SEQ ID NO: 15 - RNA, 23 nt, Homo sapiens
  misc_feature 1..23: note = hsa-miR-221-3p
  source 1..23: mol_type = genomic RNA
agctacattgtctgctgggtttc

SEQ ID NO: 16 - RNA, 23 nt, Homo sapiens
  misc_feature 1..23: note = hsa-miR-181a-5p
  source 1..23: mol_type = genomic RNA
aacattcaacgctgtcggtgagt

SEQ ID NO: 17 - RNA, 22 nt, Homo sapiens
  misc_feature 1..22: note = hsa-miR-122-5p
  source 1..22: mol_type = genomic RNA
tggagtgtgacaatggtgtttg

SEQ ID NO: 18 - RNA, 23 nt, Homo sapiens
  misc_feature 1..23: note = hsa-miR-199a-5p
  source 1..23: mol_type = genomic RNA
cccagtgttcagactacctgttc

SEQ ID NO: 19 - RNA, 22 nt, Homo sapiens
  misc_feature 1..22: note = hsa-miR-29c-3p
  source 1..22: mol_type = genomic RNA
tagcaccatttgaaatcggtta

SEQ ID NO: 20 - RNA, 21 nt, Homo sapiens
  misc_feature 1..21: note = hsa-miR-31-5p
  source 1..21: mol_type = genomic RNA
aggcaagatgctggcatagct

SEQ ID NO: 21 - RNA, 22 nt, Homo sapiens
  misc_feature 1..22: note = hsa-miR-1-3p
  source 1..22: mol_type = genomic RNA
tggaatgtaaagaagtatgtat

SEQ ID NO: 22 - RNA, 23 nt, Homo sapiens
  misc_feature 1..23: note = hsa-miR-20a-5p
  source 1..23: mol_type = genomic RNA
taaagtgcttatagtgcaggtag

SEQ ID NO: 23 - RNA, 21 nt, Homo sapiens
  misc_feature 1..21: note = hsa-miR-27a-3p
  source 1..21: mol_type = genomic RNA
ttcacagtggctaagttccgc

SEQ ID NO: 24 - RNA, 22 nt, Homo sapiens
  misc_feature 1..22: note = hsa-miR-203a-3p
  source 1..22: mol_type = genomic RNA
gtgaaatgtttaggaccactag

SEQ ID NO: 25 - RNA, 22 nt, Homo sapiens
  misc_feature 1..22: note = hsa-miR-141-3p
  source 1..22: mol_type = genomic RNA
taacactgtctggtaaagatgg

SEQ ID NO: 26 - RNA, 22 nt, Homo sapiens
  misc_feature 1..22: note = hsa-miR-200a-3p
  source 1..22: mol_type = genomic RNA
taacactgtctggtaacgatgt

SEQ ID NO: 27 - RNA, 22 nt, Homo sapiens
  misc_feature 1..22: note = hsa-miR-22-3p
  source 1..22: mol_type = genomic RNA
aagctgccagttgaagaactgt

SEQ ID NO: 28 - RNA, 21 nt, Homo sapiens
  misc_feature 1..21: note = hsa-miR-101-3p
  source 1..21: mol_type = genomic RNA
tacagtactgtgataactgaa

SEQ ID NO: 29 - RNA, 22 nt, Homo sapiens
  misc_feature 1..22: note = hsa-miR-16-5p
  source 1..22: mol_type = genomic RNA
tagcagcacgtaaatattggcg

SEQ ID NO: 30 - RNA, 24 nt, Homo sapiens
  misc_feature 1..24: note = hsa-miR-182-5p
  source 1..24: mol_type = genomic RNA
tttggcaatggtagaactcacact

SEQ ID NO: 31 - RNA, 22 nt, Homo sapiens
  misc_feature 1..22: note = hsa-miR-210-3p
  source 1..22: mol_type = genomic RNA
ctgtgcgtgtgacagcggctga

SEQ ID NO: 32 - RNA, 24 nt, Homo sapiens
  misc_feature 1..24: note = hsa-miR-125a-5p
  source 1..24: mol_type = genomic RNA
tccctgagaccctttaacctgtga

SEQ ID NO: 33 - RNA, 22 nt, Homo sapiens
  misc_feature 1..22: note = hsa-let-7a-5p
  source 1..22: mol_type = genomic RNA
tgaggtagtaggttgtatagtt

SEQ ID NO: 34 - RNA, 21 nt, Homo sapiens
  misc_feature 1..21: note = hsa-miR-23a-3p
  source 1..21: mol_type = genomic RNA
atcacattgccagggatttcc

SEQ ID NO: 35 - RNA, 23 nt, Homo sapiens
  misc_feature 1..23: note = hsa-miR-19a-3p
  source 1..23: mol_type = genomic RNA
tgtgcaaatctatgcaaaactga

SEQ ID NO: 36 - RNA, 22 nt, Homo sapiens
  misc_feature 1..22: note = hsa-miR-223-3p
  source 1..22: mol_type = genomic RNA
tgtcagtttgtcaaatacccca

SEQ ID NO: 37 - RNA, 21 nt, Homo sapiens
  misc_feature 1..21: note = hsa-miR-143-3p
  source 1..21: mol_type = genomic RNA
tgagatgaagcactgtagctc

SEQ ID NO: 38 - RNA, 22 nt, Homo sapiens
  misc_feature 1..22: note = hsa-miR-205-5p
  source 1..22: mol_type = genomic RNA
tccttcattccaccggagtctg

SEQ ID NO: 39 - RNA, 22 nt, Homo sapiens
  misc_feature 1..22: note = hsa-miR-30a-5p
  source 1..22: mol_type = genomic RNA
tgtaaacatcctcgactggaag

SEQ ID NO: 40 - RNA, 22 nt, Homo sapiens
  misc_feature 1..22: note = hsa-miR-133a-3p
  source 1..22: mol_type = genomic RNA
tttggtccccttcaaccagctg

SEQ ID NO: 41 - RNA, 22 nt, Homo sapiens
  misc_feature 1..22: note = hsa-miR-126-3p
  source 1..22: mol_type = genomic RNA
tcgtaccgtgagtaataatgcg

SEQ ID NO: 42 - RNA, 21 nt, Homo sapiens
  misc_feature 1..21: note = hsa-miR-128-3p
  source 1..21: mol_type = genomic RNA
tcacagtgaaccggtctcttt

SEQ ID NO: 43 - RNA, 21 nt, Homo sapiens
  misc_feature 1..21: note = hsa-miR-222-3p
  source 1..21: mol_type = genomic RNA
agctacatctggctactgggt

SEQ ID NO: 44 - RNA, 22 nt, Homo sapiens
  misc_feature 1..22: note = hsa-miR-214-3p
  source 1..22: mol_type = genomic RNA
acagcaggcacagacaggcagt

SEQ ID NO: 45 - RNA, 22 nt, Homo sapiens
  misc_feature 1..22: note = hsa-miR-133b
  source 1..22: mol_type = genomic RNA
tttggtccccttcaaccagcta

SEQ ID NO: 46 - RNA, 23 nt, Homo sapiens
  misc_feature 1..23: note = hsa-miR-181b-5p
  source 1..23: mol_type = genomic RNA
aacattcattgctgtcggtgggt

SEQ ID NO: 47 - RNA, 22 nt, Homo sapiens
  misc_feature 1..22: note = hsa-miR-15a-5p
  source 1..22: mol_type = genomic RNA
tagcagcacataatggtttgtg

SEQ ID NO: 48 - RNA, 23 nt, Homo sapiens
  misc_feature 1..23: note = hsa-miR-106a-5p
  source 1..23: mol_type = genomic RNA
aaaagtgcttacagtgcaggtag

SEQ ID NO: 49 - RNA, 22 nt, Homo sapiens
  misc_feature 1..22: note = hsa-miR-429
  source 1..22: mol_type = genomic RNA
taatactgtctggtaaaaccgt

SEQ ID NO: 50 - RNA, 24 nt, Homo sapiens
  misc_feature 1..24: note = hsa-miR-7-5p
  source 1..24: mol_type = genomic RNA
tggaagactagtgattttgttgtt

SEQ ID NO: 51 - RNA, 21 nt, Homo sapiens
  misc_feature 1..21: note = hsa-miR-106b-5p
  source 1..21: mol_type = genomic RNA
taaagtgctgacagtgcagat

SEQ ID NO: 52 - RNA, 23 nt, Homo sapiens
  misc_feature 1..23: note = hsa-miR-10b-5p
  source 1..23: mol_type = genomic RNA
taccctgtagaaccgaatttgtg

SEQ ID NO: 53 - RNA, 21 nt, Homo sapiens
  misc_feature 1..21: note = hsa-miR-192-5p
  source 1..21: mol_type = genomic RNA
ctgacctatgaattgacagcc

SEQ ID NO: 54 - RNA, 21 nt, Homo sapiens
  misc_feature 1..21: note = hsa-miR-195-5p
  source 1..21: mol_type = genomic RNA
tagcagcacagaaatattggc

SEQ ID NO: 55 - RNA, 23 nt, Homo sapiens
  misc_feature 1..23: note = hsa-miR-30c-5p
  source 1..23: mol_type = genomic RNA
tgtaaacatcctacactctcagc

SEQ ID NO: 56 - RNA, 23 nt, Homo sapiens
  misc_feature 1..23: note = hsa-miR-335-5p
  source 1..23: mol_type = genomic RNA
tcaagagcaataacgaaaaatgt

SEQ ID NO: 57 - RNA, 22 nt, Homo sapiens
  misc_feature 1..22: note = hsa-let-7b-5p
  source 1..22: mol_type = genomic RNA
tgaggtagtaggttgtgtggtt

SEQ ID NO: 58 - RNA, 25 nt, Homo sapiens
  misc_feature 1..25: note = hsa-miR-224-5p
  source 1..25: mol_type = genomic RNA
tcaagtcactagtggttccgtttag

SEQ ID NO: 59 - RNA, 23 nt, Homo sapiens
  misc_feature 1..23: note = hsa-miR-135a-5p
  source 1..23: mol_type = genomic RNA
tatggctttttattcctatgtga

SEQ ID NO: 60 - RNA, 22 nt, Homo sapiens
  misc_feature 1..22: note = hsa-miR-206
  source 1..22: mol_type = genomic RNA
tggaatgtaaggaagtgtgtgg

SEQ ID NO: 61 - RNA, 22 nt, Homo sapiens
  misc_feature 1..22: note = hsa-miR-92a-3p
  source 1..22: mol_type = genomic RNA
tattgcacttgtcccggcctgt

SEQ ID NO: 62 - RNA, 22 nt, Homo sapiens
  misc_feature 1..22: note = hsa-miR-150-5p
  source 1..22: mol_type = genomic RNA
tctcccaacccttgtaccagtg

SEQ ID NO: 63 - RNA, 22 nt, Homo sapiens
  misc_feature 1..22: note = hsa-miR-15b-5p
  source 1..22: mol_type = genomic RNA
tagcagcacatcatggtttaca

SEQ ID NO: 64 - RNA, 22 nt, Homo sapiens
  misc_feature 1..22: note = hsa-miR-130a-3p
  source 1..22: mol_type = genomic RNA
cagtgcaatgttaaaagggcat

SEQ ID NO: 65 - RNA, 22 nt, Homo sapiens
  misc_feature 1..22: note = hsa-miR-130b-3p
  source 1..22: mol_type = genomic RNA
cagtgcaatgatgaaagggcat

SEQ ID NO: 66 - RNA, 22 nt, Homo sapiens
  misc_feature 1..22: note = hsa-miR-140-5p
  source 1..22: mol_type = genomic RNA
cagtggttttaccctatggtag

SEQ ID NO: 67 - RNA, 23 nt, Homo sapiens
  misc_feature 1..23: note = hsa-miR-18a-5p
  source 1..23: mol_type = genomic RNA
taaggtgcatctagtgcagatag

SEQ ID NO: 68 - RNA, 22 nt, Homo sapiens
  misc_feature 1..22: note = hsa-let-7c-5p
  source 1..22: mol_type = genomic RNA
tgaggtagtaggttgtatggtt

SEQ ID NO: 69 - RNA, 22 nt, Homo sapiens
  misc_feature 1..22: note = hsa-miR-196a-5p
  source 1..22: mol_type = genomic RNA
taggtagtttcatgttgttggg

SEQ ID NO: 70 - RNA, 22 nt, Homo sapiens
  misc_feature 1..22: note = hsa-miR-199a-3p
  source 1..22: mol_type = genomic RNA
acagtagtctgcacattggtta

SEQ ID NO: 71 - RNA, 23 nt, Homo sapiens
  misc_feature 1..23: note = hsa-miR-103a-3p
  source 1..23: mol_type = genomic RNA
agcagcattgtacagggctatga

SEQ ID NO: 72 - RNA, 21 nt, Homo sapiens
  misc_feature 1..21: note = hsa-miR-129-5p
  source 1..21: mol_type = genomic RNA
ctttttgcggtctgggcttgc

SEQ ID NO: 73 - RNA, 21 nt, Homo sapiens
  misc_feature 1..21: note = hsa-miR-152-3p
  source 1..21: mol_type = genomic RNA
tcagtgcatgacagaacttgg

SEQ ID NO: 74 - RNA, 20 nt, Homo sapiens
  misc_feature 1..20: note = hsa-miR-144-3p
  source 1..20: mol_type = genomic RNA
tacagtatagatgatgtact

SEQ ID NO: 75 - RNA, 22 nt, Homo sapiens
  misc_feature 1..22: note = hsa-miR-183-5p
  source 1..22: mol_type = genomic RNA
tatggcactggtagaattcact

SEQ ID NO: 76 - RNA, 23 nt, Homo sapiens
  misc_feature 1..23: note = hsa-miR-93-5p
  source 1..23: mol_type = genomic RNA
caaagtgctgttcgtgcaggtag

SEQ ID NO: 77 - RNA, 22 nt, Homo sapiens
  misc_feature 1..22: note = hsa-miR-100-5p
  source 1..22: mol_type = genomic RNA
aacccgtagatccgaacttgtg

SEQ ID NO: 78 - RNA, 23 nt, Homo sapiens
  misc_feature 1..23: note = hsa-miR-19b-3p
  source 1..23: mol_type = genomic RNA
tgtgcaaatccatgcaaaactga

SEQ ID NO: 79 - RNA, 22 nt, Homo sapiens
  misc_feature 1..22: note = hsa-miR-30b-5p
  source 1..22: mol_type = genomic RNA
tgtaaacatcctacactcagct

SEQ ID NO: 80 - RNA, 23 nt, Homo sapiens
  misc_feature 1..23: note = hsa-miR-373-3p
  source 1..23: mol_type = genomic RNA
gaagtgcttcgattttggggtgt

SEQ ID NO: 81 - RNA, 22 nt, Homo sapiens
  misc_feature 1..22: note = hsa-miR-451a
  source 1..22: mol_type = genomic RNA
aaaccgttaccattactgagtt

SEQ ID NO: 82 - RNA, 23 nt, Homo sapiens
  misc_feature 1..23: note = hsa-miR-142-3p
  source 1..23: mol_type = genomic RNA
tgtagtgtttcctactttatgga

SEQ ID NO: 83 - RNA, 23 nt, Homo sapiens
  misc_feature 1..23: note = hsa-miR-20b-5p
  source 1..23: mol_type = genomic RNA
caaagtgctcatagtgcaggtag

SEQ ID NO: 84 - RNA, 22 nt, Homo sapiens
  misc_feature 1..22: note = hsa-miR-30d-5p
  source 1..22: mol_type = genomic RNA
tgtaaacatccccgactggaag

SEQ ID NO: 85 - RNA, 23 nt, Homo sapiens
  misc_feature 1..23: note = hsa-miR-372-3p
  source 1..23: mol_type = genomic RNA
aaagtgctgcgacatttgagcgt

SEQ ID NO: 86 - RNA, 23 nt, Homo sapiens
  misc_feature 1..23: note = hsa-miR-135b-5p
  source 1..23: mol_type = genomic RNA
tatggcttttcattcctatgtga

SEQ ID NO: 87 - RNA, 22 nt, Homo sapiens
  misc_feature 1..22: note = hsa-miR-193a-3p
  source 1..22: mol_type = genomic RNA
aactggcctacaaagtcccagt

SEQ ID NO: 88 - RNA, 22 nt, Homo sapiens
  misc_feature 1..22: note = hsa-miR-409-3p
  source 1..22: mol_type = genomic RNA
gaatgttgctcggtgaacccct

SEQ ID NO: 89 - RNA, 22 nt, Homo sapiens
  misc_feature 1..22: note = hsa-let-7g-5p
  source 1..22: mol_type = genomic RNA
tgaggtagtagtttgtacagtt

SEQ ID NO: 90 - RNA, 23 nt, Homo sapiens
  misc_feature 1..23: note = hsa-miR-10a-5p
  source 1..23: mol_type = genomic RNA
taccctgtagatccgaatttgtg

SEQ ID NO: 91 - RNA, 23 nt, Homo sapiens
  misc_feature 1..23: note = hsa-miR-191-5p
  source 1..23: mol_type = genomic RNA
caacggaatcccaaaagcagctg

SEQ ID NO: 92 - RNA, 22 nt, Homo sapiens
  misc_feature 1..22: note = hsa-let-7f-5p
  source 1..22: mol_type = genomic RNA
tgaggtagtagattgtatagtt

SEQ ID NO: 93 - RNA, 22 nt, Homo sapiens
  misc_feature 1..22: note = hsa-miR-134-5p
  source 1..22: mol_type = genomic RNA
tgtgactggttgaccagagggg

SEQ ID NO: 94 - RNA, 23 nt, Homo sapiens
  misc_feature 1..23: note = hsa-miR-146b-5p
  source 1..23: mol_type = genomic RNA
tgagaactgaattccataggctg

SEQ ID NO: 95 - RNA, 22 nt, Homo sapiens
  misc_feature 1..22: note = hsa-miR-127-3p
  source 1..22: mol_type = genomic RNA
tcggatccgtctgagcttggct

SEQ ID NO: 96 - RNA, 22 nt, Homo sapiens
  misc_feature 1..22: note = hsa-miR-196b-5p
  source 1..22: mol_type = genomic RNA
taggtagtttcctgttgttggg

SEQ ID NO: 97 - RNA, 23 nt, Homo sapiens
  misc_feature 1..23: note = hsa-miR-302d-3p
  source 1..23: mol_type = genomic RNA
taagtgcttccatgtttgagtgt

SEQ ID NO: 98 - RNA, 22 nt, Homo sapiens
  misc_feature 1..22: note = hsa-miR-663a
  source 1..22: mol_type = genomic RNA
aggcggggcgccgcgggaccgc

SEQ ID NO: 99 - RNA, 20 nt, Homo sapiens
  misc_feature 1..20: note = hsa-miR-326
  source 1..20: mol_type = genomic RNA
cctctgggcccttcctccag

SEQ ID NO: 100 - RNA, 22 nt, Homo sapiens
  misc_feature 1..22: note = hsa-miR-486-5p
  source 1..22: mol_type = genomic RNA
tcctgtactgagctgccccgag

SEQ ID NO: 101 - RNA, 22 nt, Homo sapiens
  misc_feature 1..22: note = hsa-miR-17-3p
  source 1..22: mol_type = genomic RNA
actgcagtgaaggcacttgtag

SEQ ID NO: 102 - RNA, 22 nt, Homo sapiens
  misc_feature 1..22: note = hsa-miR-30e-5p
  source 1..22: mol_type = genomic RNA
tgtaaacatccttgactggaag

SEQ ID NO: 103 - RNA, 22 nt, Homo sapiens
  misc_feature 1..22: note = hsa-let-7d-5p
  source 1..22: mol_type = genomic RNA
agaggtagtaggttgcatagtt

SEQ ID NO: 104 - RNA, 22 nt, Homo sapiens
  misc_feature 1..22: note = hsa-miR-193b-3p
  source 1..22: mol_type = genomic RNA
aactggccctcaaagtcccgct

SEQ ID NO: 105 - RNA, 20 nt, Homo sapiens
  misc_feature 1..20: note = hsa-miR-202-3p
  source 1..20: mol_type = genomic RNA
agaggtatagggcatgggaa

SEQ ID NO: 106 - RNA, 22 nt, Homo sapiens
  misc_feature 1..22: note = hsa-miR-216a-5p
  source 1..22: mol_type = genomic RNA
taatctcagctggcaactgtga

SEQ ID NO: 107 - RNA, 21 nt, Homo sapiens
  misc_feature 1..21: note = hsa-miR-376c-3p
  source 1..21: mol_type = genomic RNA
aacatagaggaaattccacgt

SEQ ID NO: 108 - RNA, 22 nt, Homo sapiens
  misc_feature 1..22: note = hsa-miR-198
  source 1..22: mol_type = genomic RNA
ggtccagaggggagataggttc

SEQ ID NO: 109 - RNA, 21 nt, Homo sapiens
  misc_feature 1..21: note = hsa-miR-215-5p
  source 1..21: mol_type = genomic RNA
atgacctatgaattgacagac

SEQ ID NO: 110 - RNA, 22 nt, Homo sapiens
  misc_feature 1..22: note = hsa-miR-197-3p
  source 1..22: mol_type = genomic RNA
ttcaccaccttctccacccagc

SEQ ID NO: 111 - RNA, 22 nt, Homo sapiens
  misc_feature 1..22: note = hsa-miR-29a-5p
  source 1..22: mol_type = genomic RNA
actgatttcttttggtgttcag

SEQ ID NO: 112 - RNA, 23 nt, Homo sapiens
  misc_feature 1..23: note = hsa-miR-425-5p
  source 1..23: mol_type = genomic RNA
aatgacacgatcactcccgttga

SEQ ID NO: 113 - RNA, 22 nt, Homo sapiens
  misc_feature 1..22: note = hsa-miR-574-3p
  source 1..22: mol_type = genomic RNA
cacgctcatgcacacacccaca

SEQ ID NO: 114 - RNA, 23 nt, Homo sapiens
  misc_feature 1..23: note = hsa-miR-18b-5p
  source 1..23: mol_type = genomic RNA
taaggtgcatctagtgcagttag

SEQ ID NO: 115 - RNA, 22 nt, Homo sapiens
  misc_feature 1..22: note = hsa-miR-483-5p
  source 1..22: mol_type = genomic RNA
aagacgggaggaaagaagggag

SEQ ID NO: 116 - RNA, 21 nt, Homo sapiens
  misc_feature 1..21: note = hsa-miR-625-5p
  source 1..21: mol_type = genomic RNA
agggggaaagttctatagtcc

SEQ ID NO: 117 - RNA, 22 nt, Homo sapiens
  misc_feature 1..22: note = hsa-miR-338-5p
  source 1..22: mol_type = genomic RNA
aacaatatcctggtgctgagtg

SEQ ID NO: 118 - RNA, 22 nt, Homo sapiens
  misc_feature 1..22: note = hsa-miR-539-5p
  source 1..22: mol_type = genomic RNA
ggagaaattatccttggtgtgt

SEQ ID NO: 119 - RNA, 21 nt, Homo sapiens
  misc_feature 1..21: note = hsa-miR-151a-3p
  source 1..21: mol_type = genomic RNA
ctagactgaagctccttgagg

SEQ ID NO: 120 - RNA, 22 nt, Homo sapiens
  misc_feature 1..22: note = hsa-miR-208b-3p
  source 1..22: mol_type = genomic RNA
ataagacgaacaaaaggtttgt

SEQ ID NO: 121 - RNA, 22 nt, Homo sapiens
  misc_feature 1..22: note = hsa-miR-330-5p
  source 1..22: mol_type = genomic RNA
tctctgggcctgtgtcttaggc

SEQ ID NO: 122 - RNA, 22 nt, Homo sapiens
  misc_feature 1..22: note = hsa-miR-382-5p
  source 1..22: mol_type = genomic RNA
gaagttgttcgtggtggattcg

SEQ ID NO: 123 - RNA, 21 nt, Homo sapiens
  misc_feature 1..21: note = hsa-miR-499a-5p
  source 1..21: mol_type = genomic RNA
ttaagacttgcagtgatgttt

SEQ ID NO: 124 - RNA, 22 nt, Homo sapiens
  misc_feature 1..22: note = hsa-miR-223-5p
  source 1..22: mol_type = genomic RNA
cgtgtatttgacaagctgagtt

SEQ ID NO: 125 - RNA, 22 nt, Homo sapiens
  misc_feature 1..22: note = hsa-miR-31-3p
  source 1..22: mol_type = genomic RNA
tgctatgccaacatattgccat

SEQ ID NO: 126 - RNA, 22 nt, Homo sapiens
  misc_feature 1..22: note = hsa-miR-361-5p
  source 1..22: mol_type = genomic RNA
ttatcagaatctccaggggtac

SEQ ID NO: 127 - RNA, 23 nt, Homo sapiens
  misc_feature 1..23: note = hsa-miR-423-3p
  source 1..23: mol_type = genomic RNA
agctcggtctgaggcccctcagt

SEQ ID NO: 128 - RNA, 22 nt, Homo sapiens
  misc_feature 1..22: note = hsa-miR-885-5p
  source 1..22: mol_type = genomic RNA
tccattacactaccctgcctct

SEQ ID NO: 129 - RNA, 22 nt, Homo sapiens
  misc_feature 1..22: note = hsa-miR-95-3p
  source 1..22: mol_type = genomic RNA
ttcaacgggtatttattgagca

SEQ ID NO: 130 - RNA, 22 nt, Homo sapiens
  misc_feature 1..22: note = hsa-miR-99b-5p
  source 1..22: mol_type = genomic RNA
cacccgtagaaccgaccttgcg

SEQ ID NO: 131 - RNA, 22 nt, Homo sapiens
  misc_feature 1..22: note = hsa-miR-299-5p
  source 1..22: mol_type = genomic RNA
tggtttaccgtcccacatacat

SEQ ID NO: 132 - RNA, 22 nt, Homo sapiens
  misc_feature 1..22: note = hsa-miR-378a-5p
  source 1..22: mol_type = genomic RNA
ctcctgactccaggtcctgtgt

SEQ ID NO: 133 - RNA, 23 nt, Homo sapiens
  misc_feature 1..23: note = hsa-miR-500a-5p
  source 1..23: mol_type = genomic RNA
taatccttgctacctgggtgaga

SEQ ID NO: 134 - RNA, 20 nt, Homo sapiens
  misc_feature 1..20: note = hsa-miR-518a-5p
  source 1..20: mol_type = genomic RNA
ctgcaaagggaagccctttc

SEQ ID NO: 135 - RNA, 22 nt, Homo sapiens
  misc_feature 1..22: note = hsa-miR-589-5p
  source 1..22: mol_type = genomic RNA
tgagaaccacgtctgctctgag

SEQ ID NO: 136 - RNA, 21 nt, Homo sapiens
  misc_feature 1..21: note = hsa-miR-718
  source 1..21: mol_type = genomic RNA
cttccgccccgccgggcgtcg

SEQ ID NO: 137 - RNA, 21 nt, Homo sapiens
  misc_feature 1..21: note = hsa-miR-940
  source 1..21: mol_type = genomic RNA
aaggcagggcccccgctcccc

SEQ ID NO: 138 - RNA, 22 nt, Homo sapiens
  misc_feature 1..22: note = hsa-miR-28-3p
  source 1..22: mol_type = genomic RNA
cactagattgtgagctcctgga

SEQ ID NO: 139 - RNA, 21 nt, Homo sapiens
  misc_feature 1..21: note = hsa-miR-411-5p
  source 1..21: mol_type = genomic RNA
tagtagaccgtatagcgtacg

SEQ ID NO: 140 - RNA, 23 nt, Homo sapiens
  misc_feature 1..23: note = hsa-miR-423-5p
  source 1..23: mol_type = genomic RNA
tgaggggcagagagcgagacttt

SEQ ID NO: 141 - RNA, 22 nt, Homo sapiens
  misc_feature 1..22: note = hsa-miR-450a-5p
  source 1..22: mol_type = genomic RNA
ttttgcgatgtgttcctaatat

SEQ ID NO: 142 - RNA, 22 nt, Homo sapiens
  misc_feature 1..22: note = hsa-miR-484
  source 1..22: mol_type = genomic RNA
tcaggctcagtcccctcccgat

SEQ ID NO: 143 - RNA, 25 nt, Homo sapiens
  misc_feature 1..25: note = hsa-miR-593-5p
  source 1..25: mol_type = genomic RNA
aggcaccagccaggcattgctcagc

SEQ ID NO: 144 - RNA, 21 nt, Homo sapiens
  misc_feature 1..21: note = hsa-miR-652-3p
  source 1..21: mol_type = genomic RNA
aatggcgccactagggttgtg

SEQ ID NO: 145 - RNA, 20 nt, Homo sapiens
  misc_feature 1..20: note = hsa-miR-760
  source 1..20: mol_type = genomic RNA
cggctctgggtctgtgggga

SEQ ID NO: 146 - RNA, 21 nt, Homo sapiens
  misc_feature 1..21: note = hsa-miR-1228-5p
  source 1..21: mol_type = genomic RNA
gtgggcgggggcaggtgtgtg

SEQ ID NO: 147 - RNA, 24 nt, Homo sapiens
  misc_feature 1..24: note = hsa-miR-1254
  source 1..24: mol_type = genomic RNA
agcctggaagctggagcctgcagt

SEQ ID NO: 148 - RNA, 19 nt, Homo sapiens
  misc_feature 1..19: note = hsa-miR-1290
  source 1..19: mol_type = genomic RNA
tggatttttggatcaggga

SEQ ID NO: 149 - RNA, 23 nt, Homo sapiens
  misc_feature 1..23: note = hsa-miR-574-5p
  source 1..23: mol_type = genomic RNA
tgagtgtgtgtgtgtgagtgtgt

SEQ ID NO: 150 - RNA, 23 nt, Homo sapiens
  misc_feature 1..23: note = hsa-miR-579-3p
  source 1..23: mol_type = genomic RNA
ttcatttggtataaaccgcgatt

SEQ ID NO: 151 - RNA, 21 nt, Homo sapiens
  misc_feature 1..21: note = hsa-miR-596
  source 1..21: mol_type = genomic RNA
aagcctgcccggctcctcggg

SEQ ID NO: 152 - RNA, 22 nt, Homo sapiens
  misc_feature 1..22: note = hsa-miR-601
  source 1..22: mol_type = genomic RNA
tggtctaggattgttggaggag

SEQ ID NO: 153 - RNA, 22 nt, Homo sapiens
  misc_feature 1..22: note = hsa-miR-660-5p
  source 1..22: mol_type = genomic RNA
tacccattgcatatcggagttg

SEQ ID NO: 154 - RNA, 22 nt, Homo sapiens
  misc_feature 1..22: note = hsa-let-7d-3p
  source 1..22: mol_type = genomic RNA
ctatacgacctgctgcctttct

SEQ ID NO: 155 - RNA, 22 nt, Homo sapiens
  misc_feature 1..22: note = hsa-miR-1225-3p
  source 1..22: mol_type = genomic RNA
tgagcccctgtgccgcccccag

SEQ ID NO: 156 - RNA, 27 nt, Homo sapiens
  misc_feature 1..27: note = hsa-miR-1248
  source 1..27: mol_type = genomic RNA
accttcttgtataagcactgtgctaaa

SEQ ID NO: 157 - RNA, 22 nt, Homo sapiens
  misc_feature 1..22: note = hsa-miR-1972
  source 1..22: mol_type = genomic RNA
tcaggccaggcacagtggctca

SEQ ID NO: 158 - RNA, 19 nt, Homo sapiens
  misc_feature 1..19: note = hsa-miR-1973
  source 1..19: mol_type = genomic RNA
accgtgcaaaggtagcata

SEQ ID NO: 159 - RNA, 21 nt, Homo sapiens
  misc_feature 1..21: note = hsa-miR-2114-3p
  source 1..21: mol_type = genomic RNA
cgagcctcaagcaagggactt

SEQ ID NO: 160 - RNA, 23 nt, Homo sapiens
  misc_feature 1..23: note = hsa-miR-217-5p
  source 1..23: mol_type = genomic RNA
tactgcatcaggaactgattgga

SEQ ID NO: 161 - RNA, 22 nt, Homo sapiens
  misc_feature 1..22: note = hsa-miR-320a-3p
  source 1..22: mol_type = genomic RNA
aaaagctgggttgagagggcga

SEQ ID NO: 162 - RNA, 22 nt, Homo sapiens
  misc_feature 1..22: note = hsa-miR-375-3p
  source 1..22: mol_type = genomic RNA
tttgttcgttcggctcgcgtga

SEQ ID NO: 163 - RNA, 22 nt, Homo sapiens
  misc_feature 1..22: note = hsa-miR-425-3p
  source 1..22: mol_type = genomic RNA
atcgggaatgtcgtgtccgccc

SEQ ID NO: 164 - RNA, 17 nt, Homo sapiens
  misc_feature 1..17: note = hsa-miR-4306
  source 1..17: mol_type = genomic RNA
tggagagaaaggcagta

SEQ ID NO: 165 - RNA, 22 nt, Homo sapiens
  misc_feature 1..22: note = hsa-miR-452-3p
  source 1..22: mol_type = genomic RNA
ctcatctgcaaagaagtaagtg

SEQ ID NO: 166 - RNA, 22 nt, Homo sapiens
  misc_feature 1..22: note = hsa-miR-4772-3p
  source 1..22: mol_type = genomic RNA
cctgcaactttgcctgatcaga

SEQ ID NO: 167 - RNA, 21 nt, Homo sapiens
  misc_feature 1..21: note = hsa-miR-520b-3P
  source 1..21: mol_type = genomic RNA
aaagtgcttccttttagaggg

SEQ ID NO: 168 - DNA, 24 bp, synthetic construct
  misc_feature 1..24: note = Synthetic
  source 1..24: mol_type = other DNA
caactcaggtcgtaggcaattcgt